(19) United States
(12) Patent Application Publication
(10) Pub. No.: US 2023/0218649 A1
(43) Pub. Date: Jul. 13, 2023

(54) COMPOSITIONS AND METHODS FOR OVERCOMING MICROENVIRONMENT-MEDIATED RESISTANCE VIA E-SELECTIN TARGETING

(71) Applicant: GLYCOMIMETICS, INC., Rockville, MD (US)

(72) Inventors: John L. MAGNANI, Gaithersburg, MD (US); William E. FOGLER, Lutherville-Timonium, MD (US); Theodore SMITH, Ijamsville, MD (US)

(73) Assignee: GLYCOMIMETICS, INC., Rockville, MD (US)

(21) Appl. No.: 18/001,614

(22) PCT Filed: Jun. 11, 2021
(86) PCT No.: PCT/US2021/036992

§ 371 Date: Dec. 13, 2022

Related U.S. Application Data
(60) Provisional application No. 63/038,856, filed on Jun. 14, 2020.
(60) Provisional application No. 63/060,605, filed on Aug. 3, 2020.
(60) Provisional application No. 63/198,856, filed on Nov. 17, 2020.

(51) Int. Cl. A61K 31/7034; A61K 31/496; A61P 35/02; A61K 31/706

(52) U.S. Cl. CPC A61K 31/7034; A61K 31/496; A61P 35/02; A61K 31/706

(57) ABSTRACT

Methods for treating a cancer (such as, e.g., acute myeloid leukemia) comprising administering to a subject (such as, e.g., a subject who has acquired resistance to a therapy comprising at least one antineoplastic agent and/or at least one hypomethylating agent) at least one E-selectin antagonist, wherein the subject is further administered at least one antineoplastic agent (such as, e.g., venetoclax) and/or at least one hypomethylating agent are disclosed.

This application claims the benefit of priority of U.S. Provisional Application No. 63/038,856, filed Jun. 14, 2020, U.S. Provisional Application No. 63/060,605, filed Aug. 3, 2020, and U.S. Provisional Application No. 63/198,856, filed Nov. 17, 2020, the contents of each of which are herein incorporated by reference in their entirety.

Disclosed herein are methods of treating a cancer (such as, e.g., acute myeloid leukemia (AML)) in a subject in need thereof comprising administering to the subject at least one E-selectin antagonist, wherein the subject is further administered at least one antineoplastic agent (such as, e.g., venetoclax) and/or at least one hypomethylating agent. In some embodiments, the subject is a relapsed cancer patient. In some embodiments, the subject has acquired resistance to a therapy comprising the at least one antineoplastic agent and/or the at least one hypomethylating agent. In some embodiments, blast cells in the subject have an increased gene expression level of FUT7 and/or ST3GAL4 relative to a control sample from a non-cancer subject, a newly diagnosed cancer subject, or a subject having the same cancer as the patient.

Selectins are a class of cell adhesion molecules that have well-characterized roles in leukocyte homing. These cell-adhesion molecules are type 1 membrane proteins and are composed of an amino terminal lectin domain, an epidermal growth factor (EGF)-like domain, a variable number of complement receptor related repeats, a hydrophobic domain spanning region, and a cytoplasmic domain. Binding interactions appear to be mediated by contact of the lectin domain of the selectins and various carbohydrate ligands.

There are three known selectins: E-selectin; P-selectin; and L-selectin. The vascular adhesion molecule E-selectin is expressed by endothelial cells in response to IL-1, lipopolysaccharide, TNF-α, or IFNγ (Bevilacqua et al., 1987), and deletion or blockade of E-selectin promotes hematopoietic stem cell (HSC) quiescence, self-renewal potential, and chemoresistance (Winkler et al., 2012). E-selectin is a transmembrane adhesion protein expressed on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged. Specifically, E-selectin is responsible for the tethering and rolling of leukocytes on perivascular endothelial bone marrow niche cells. In addition, E-selectin binds to sialyl-Lewis^{a} (sLe^{a}), which is expressed on many tumor cells. In leukemia, E-selectin and its ligand binding have crucial roles in bone marrow homing and engraftment (Krause et al., 2006).

P-selectin is expressed on inflamed endothelium and platelets and also recognizes sLe^{x} and sLe^{a}; however, P-selectin contains a second site that interacts with sulfated tyrosine. The expression of E-selectin and P-selectin is generally increased when the tissue adjacent to a capillary is infected or damaged. L-selectin is expressed on leukocytes.

Many cancers are treatable before the cancer has moved beyond the primary site. However, once the cancer has spread beyond the primary site, the treatment options may be limited and the survival statistics may decline dramatically. Recent investigations have suggested that cancer cells are immunostimulatory and interact with selectins to extravasate and metastasize.

Based on estimated incidence data, the most common types of cancer include prostate, breast, lung, colorectal, melanoma, bladder, non-Hodgkin's lymphoma, kidney, thyroid, leukemias, endometrial, and pancreatic cancers. The cancer with the highest expected incidence is prostate cancer. The highest mortality rate is for patients who have lung cancer. Despite enormous investments of financial and human resources, cancers such as colorectal cancer remain a leading cause of death. Illustratively, colorectal cancer is the second leading cause of cancer-related deaths in the United States among cancers that affect both men and women. Over the last several years, more than 50,000 patients with colorectal cancer have died annually.

The four most common hematological cancers are acute lymphocytic leukemia (ALL), chronic lymphocytic leukemia (CLL), chronic myelogenous leukemia (CML) and acute myelogenous leukemia (AML). Leukemias and other cancers of the blood, bone marrow, and lymphatic system affect 10 times more adults than children. However, leukemia is one of the most common childhood cancers, and 75% of childhood leukemias are ALL.

Acute myeloid leukemia (AML) is an aggressive, heterogeneous hematologic disease characterized by the rapid growth of abnormal progenitors (blasts) in the bone marrow and blood, which interferes with normal blood cell production. AML is the most common leukemia in adults, and the incidence of AML has been increasing in recent years. More than 300,000 people in the world are diagnosed with AML annually, and over 150,000 deaths due to AML are reported each year. The median age at diagnosis is 66 years, with cure rates of less than 10% and median survival of less than 1 year (Burnett et al., 2010). Although 70-80% of patients younger than 60 years achieve complete remission, most eventually relapse, and overall survival is only 40-50% at 5 years (Fernandez et al., 2009; Mandelli et al., 2009; Ravandi et al., 2006).

AML can progress rapidly and is typically fatal within weeks or months if left untreated. AML symptoms may include fatigue, shortness of breath, easy bruising and bleeding, and increased risk of infection. First-line treatment of AML consists primarily of chemotherapy with an anthracycline/cytarabine or daunorubicin/cytarabine combination and is divided into two phases: induction and post-remission (or consolidation) therapy. The goal of induction therapy is to achieve a complete remission by reducing the number of leukemic cells to an undetectable level, while the goal of consolidation therapy is to eliminate any residual undetectable disease and achieve a cure. The specific genetic mutations present within the cancer cells may guide therapy, as well as determine how long a patient is likely to survive.

Although intensive chemotherapy is the standard of care for younger AML patients, elderly patients are often susceptible to treatment-related morbidity and mortality. Recently, the hypomethylating agents (HMAs) azacitidine and decitabine in combination with low-dose cytarabine have been used to treat patients who are not eligible for intensive chemotherapy. More recently, clinical studies have demonstrated that combinations of the FDA-approved Bcl-2 inhibitor venetoclax and hypomethylating agents are highly effective in elderly patients with AML (DiNardo et al., 2019).

Despite these advances, the duration of response is still short, and median survival remains unsatisfactory for most patients. The majority of patients who achieve complete remission (CR) following induction therapy will relapse within three years of diagnosis. The prognosis is extremely poor for AML patients who have experienced relapse.

Accordingly, there is a need for novel methods of treating cancer, such as, e.g., AML, including novel methods for overcoming microenvironment-mediated resistance to antineoplastic agents.

Recently, various relapse mechanisms have been studied extensively, and the primary cause of treatment failure in AML patients is now thought to be the survival of therapy-resistant leukemic stem cells (LSC) in the bone marrow (BM) microenvironment (Konopleva & Jordan, 2011) and elevated alternative anti-apoptotic protein, Mcl-1 (Konopleva et al., 2016).

The bone marrow microenvironment plays a critical role in leukemia initiation, progression, and drug resistance. Adhesion to the bone marrow niche is critical for AML initiation and progression and LSC survival after induction therapy, which contributes to subsequent relapse. Illustratively, AML cells residing in bone marrow receive a great deal of protection from the cytotoxic effects of chemotherapeutic agents. In contrast, circulating leukemia cells are typically more chemo-sensitive compared to those embedded in bone marrow niches. The bone marrow homing of AML cells is mediated by multiple adhesive and chemokinetic interactions including, respectively, by sialylated glycoproteins on the cancer cells binding to E-selectin on the endothelium.

The Fms-like tyrosine kinase 3 (FLT3-ITD) mutation in AML patients is significantly associated with the expression of E-selectin (Kupsa et al., 2016). Specifically, the correlation of higher E-selectin expression in patients containing the FLT3-ITD mutation in their AML cells is strongly significant (p=0.0010) (Kupsa et al., 2016). Internal tandem duplications in the FLT3-ITD account for 30% of adult AML cases and confer poor prognosis (Nakao et al., 1996; Kottaridis et al., 2003; Thiede et al., 2002). The hallmark of AML cells containing mutations in the FLT3 gene is the constitutive kinase activation of these cancer cells.

Gene expression of FUT7, an E-selectin ligand glycosylation gene, correlates to expression of the E-selectin ligand (sialyl Le^{x}) on the surface of AML cells in patients. FUT7 codes for the fucosyltransferase that adds the terminal fucose required for binding activity of the E-selectin ligand. In an analysis of a public database of AML patients, which is known as TCGA (The Cancer Genome Atlas) from NCI containing 151 paired data with Overall Survival, poor survival was only observed in FLT3-ITD AML patients that express the E-selectin ligand as determined by FUT7 expression. (See PCT International Publication No. WO 2021/011435, which is incorporated by reference herein.) Correlation of poor survival with expression of the E-selectin ligand as determined by FUT7 expression in FLT3-ITD patients is statistically significant (p=0.015), suggesting that the binding of AML cells to E-selectin drives the poor survival observed with AML patients with FLT3 mutations. Additionally, AML patients harboring the FLT3 ITD mutation with high expressions of FUT7 and ST3GAL4, another E-selectin ligand-forming glycosylation gene, experience poor survival compared to patients with low expression of FUT7 and ST3GAL4. (See PCT International Publication No. WO 2021/011435.)

Elevated soluble E-selectin levels have also been detected in relapsed AML (Aref et al., 2002). Adhesion to E-selectin leads to chemoresistance and likely contributes to subsequent relapse. In studies described herein, the roles of E-selectin in AML survival using human AML cell lines and patient-derived AML xenograft (PDX) models were elucidated. In the reported experiments, E-selectin binding decreased expression of CDK4 and CDK6, and increased dormancy of AML cells in vitro. Additionally, targeting E-selectin mobilized human AML cells and sensitized them to venetoclax/HMA therapy.

Thus, administration of an E-selectin antagonist in combination with an antineoplastic agent (such as, e.g., venetoclax) and/or a hypomethylating agent may be useful for overcoming microenvironment-mediated resistance to chemotherapy and/or for treating cancer (such as, e.g., AML). E-selectin antagonists like Compound A, which interrupt leukemic cell homing to the vascular niche, increase susceptibility to cytotoxic and targeted therapies and can be potent adjuncts to antineoplastic agents and/or HMAs.

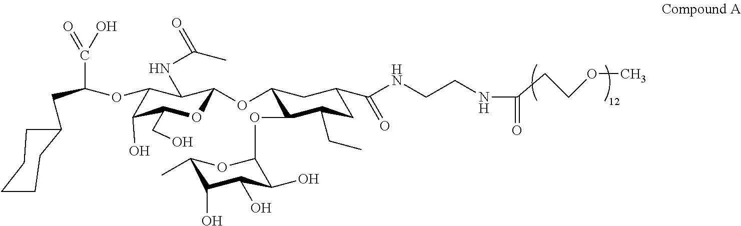

Compound A mimics the bioactive conformation of sLe^{a/x} and binds to E-selectin with high affinity (K_{D}˜0.45 μM). Pharmacological inhibition of E-selectin by Compound A increased the expression of cell cycle regulating proteins including CDK4, CDK6, CyclinD1, and CyclinD2 in HUVEC co-cultured AML.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description.

It should be understood that references herein to methods of treatment (e.g., methods of treating a cancer, such as, e.g., AML) in a subject using at least one E-selectin antagonist, wherein the subject is further administered at least one antineoplastic agent (such as, e.g., venetoclax) and/or at least one hypomethylating agent should also be interpreted as references to:
at least one E-selectin antagonist and at least one antineoplastic agent (such as, e.g., venetoclax) and/or at least one hypomethylating agent for use in methods of treating, e.g., a cancer, such as, e.g., AML, in a subject; and/or
at least one E-selectin antagonist for use in methods of treating, e.g., a cancer, such as, e.g., AML, in a subject, wherein the subject is further administered at least one antineoplastic agent (such as, e.g., venetoclax) and/or at least one hypomethylating agent; and/or
the use of at least one E-selectin antagonist and at least one antineoplastic agent (such as, e.g., venetoclax) and/or at least one hypomethylating agent in the manufacture of a medicament for treating, e.g., a cancer, such as, e.g., AML, in a subject; and/or
the use of at least one E-selectin antagonist in the manufacture of a medicament for treating, e.g., a cancer, such as, e.g., AML, in a subject, wherein the subject is further administered at least one antineoplastic agent (such as, e.g., venetoclax) and/or at least one hypomethylating agent.

### BRIEF DESCRIPTION OF THE DRAWINGS

FIG. 1 is a schematic illustrating an in vivo PDX-AML (Ven/HMA-resistant) model derived from an AML patient harboring FLT3-ITD, NRAS, and GATA2 mutations who initially responded to venetoclax/HMA therapy and then relapsed.

FIG. 2 is a diagram illustrating Kaplan-Meier survival curves of AML-PDX mice treated with Compound A, venetoclax/HMA, or a combination.

FIG. 3 is a chart showing the percentage of human CD45⁺ cells in peripheral blood circulation of mice during three weeks of treatment with vehicle control, Compound A, venetoclax/HMA, or a combination.

FIG. 4 is a chart showing the absolute number of human CD45⁺ cells in peripheral blood circulation of mice during three weeks of treatment with vehicle control, Compound A, venetoclax/HMA, or a combination.

FIG. 5 depicts representative histological images of the bone marrow, spleen, lung, and liver for normal NSC control mice and NSC mice injected with leukemia cell infiltrates then treated with vehicle control, Compound A, venetoclax/HMA, or a combination.

FIG. 6A is a t-Distributed Stochastic Neighbor Embedding (TSNE) plot depicting single cell proteomics results using CyTOF for all clusters of human CD45⁺ cells.

FIG. 6B is a TSNE plot depicting single cell proteomics results using CyTOF for cells isolated from mice following three weeks of treatment with vehicle control, Compound A, venetoclax/HMA, or a combination.

FIG. 7A is a TSNE plot depicting E-selectin ligand expression for all clusters of human CD45⁺ cells, as assessed by single cell proteomics (CyTOF).

FIG. 7B is a TSNE plot depicting E-selectin ligand expression as assessed by CyTOF for cells isolated from mice following three weeks of treatment with vehicle control, Compound A, venetoclax/HMA, or a combination.

FIG. 8A is a heatmap showing E-selectin ligand and Bcl-2 levels in mice following three weeks of treatment with vehicle control, Compound A, venetoclax/HMA, or a combination. For each annotated phenotype, median intensity of the marker expression was computed for each treatment group and visualized in heatmaps to illustrate the differences in protein expression. The scale is the mean intensity of arcsinh-transformed values.

FIG. 8B is a heatmap showing c-Myc, Ki67, and IdU levels in mice following three weeks of treatment with vehicle control, Compound A, venetoclax/HMA, or a combination. For each annotated phenotype, median intensity of the marker expression was computed for each treatment group and visualized in heatmaps to illustrate the differences in protein expression. The scale is the mean intensity of arcsinh-transformed values.

FIGS. 9A-C depicts single cell proteomics heatmaps demonstrating that E-selectin inhibition alters the proliferation of AML blasts and AML pro-survival signaling signatures.

FIG. 10 depicts single cell proteomics results (left: UMAP results; right: heatmaps) indicating that E-selectin inhibition mediates signaling alterations in the AML BM microenvironment.

FIG. 11 is a diagram illustrating Kaplan-Meier survival curves in a KG1 AML model for mice treated with saline, 5-azacitidine alone, Compound A alone, or 5-azacitidine in combination with Compound A.

FIG. 12A depicts representative immunofluorescence images of adhesion of 5-azacitidine treated KG1 cells to E-selectin.

FIG. 12B depicts a chart quantifying the adhesion of 5-azacitidine treated KG1 cells to E-selectin using fluorescence measurements.

FIG. 13 is a chart depicting flow cytometry analysis results for PE-conjugated E-selectin binding to KG1 cells.

FIG. 14 is a chart depicting the effects of 5-azacitidine on global DNA methylation in KG1 cells.

FIG. 15 is a chart depicting the results of FUT7 promoter methylation analysis for KG1 cells cultured in the presence of various concentrations of 5-azacitidine.

FIG. 16 is a diagram illustrating Kaplan-Meier survival curves in a MV4.11 AML model for mice treated with saline, venetoclax alone, Compound A alone, or venetoclax in combination with Compound A.

### DEFINITIONS

Unless otherwise defined, all technical and scientific terms used herein have the same meaning as commonly understood by one of ordinary skill in the art to which this disclosure belongs. All references cited herein are incorporated by reference in their entireties. To the extent terms or discussion in references conflict with this disclosure, the latter shall control.

Whenever a term in the specification is identified as a range (e.g., C₁₋₄ alkyl) or “ranging from,” the range independently discloses and includes each element of the range. As a non-limiting example, C₁₋₄ alkyl groups include, independently, C₁ alkyl groups, C₂ alkyl groups, C₃ alkyl groups, and C₄ alkyl groups. As another non-limiting example, “n is an integer ranging from 0 to 2” includes, independently, 0, 1, and 2.

As used herein, the singular forms of a word also include the plural form of the word, unless the context clearly dictates otherwise. For example, as used herein, “a” or “an” entity refers to one or more of that entity, e.g., “a compound” refers to one or more compounds or at least one compound unless stated otherwise. As such, the terms “a” (or “an”), “one or more,” and “at least one” are used interchangeably herein. For example, the term “at least one C₁₋₄ alkyl group” refers to one or more C₁₋₄ alkyl groups, such as one C₁₋₄ alkyl group, two C₁₋₄ alkyl groups, etc.

As used herein, the term “or” means “and/or” unless the specific context indicates otherwise.

As used herein, the term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted. Non-limiting examples of substituted alkyl groups include deuterated alkyl groups such as, e.g., CD₃ and CD₂CD₃.

As used herein, the term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

As used herein, the term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

As used herein, the term “aryl” includes hydrocarbon ring system groups comprising at least 6 carbon atoms and at least one aromatic ring. The aryl group may be a monocyclic, bicyclic, tricyclic, or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

As used herein, the term “halo” or “halogen” includes fluoro, chloro, bromo, and iodo.

As used herein, the term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. For example, a “fluoroalkyl” is a haloalkyl wherein at least one halogen is fluoro. Unless stated otherwise specifically in the specification, a haloalkyl group may be optionally substituted.

As used herein, the term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

As used herein, the term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

As used herein, the term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon, or sulfur atom(s) in the heterocyclyl group may be optionally oxidized; any nitrogen atom in the heterocyclyl group may be optionally quaternized. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

As used herein, the term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic, or tetracyclic ring system, which may include fused or bridged ring systems; and the nitrogen, carbon, or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

Unless stated otherwise specifically in the specification, substituents may be optionally substituted.

The term “substituted” includes the situation where, in any of the above groups, at least one hydrogen atom is replaced by a non-hydrogen atom such as, for example, a deuterium atom; a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

The terms “acute myeloid leukemia,” “acute myelogenous leukemia,” “acute myeloblastic leukemia,” “acute granulocytic leukemia,” and “acute nonlymphocytic leukemia,” and “AML” are used interchangeably and, as used herein, refer to a cancer of the bone marrow characterized by abnormal proliferation of myeloid stem cells. AML, as used herein, refers to any or all known subtypes of the disease, including but not limited to, subtypes classified by the World Health Organization (WHO) 2016 classification of AML, e.g., AML with myelodysplasia-related changes or myeloid sarcoma, and the French-American-British (FAB) classification system, e.g., M0 (acute myeloblastic leukemia, minimally differentiated) or M1 (acute myeloblastic leukemia, without maturation) (Falini et al., 2010; Lee et al., 1987).

As used herein, “administration” of a compound to a patient refers to any route (e.g., oral delivery) of introducing or delivering the active pharmaceutical ingredient to the patient. Administration includes self-administration and the administration by another.

As used herein, the terms “in combination with” and “is further administered,” when referring to two or more compounds, agents, or additional active pharmaceutical ingredients, means the administration of two or more compounds, agents, or active pharmaceutical ingredients to the patient prior to, concurrently with, or subsequent to each other. The two or more compounds, agents, or active pharmaceutical ingredients may be administered in the same pharmaceutical composition or different pharmaceutical compositions.

As used herein, the term “antineoplastic agent” refers to an active pharmaceutical ingredient that prevents, inhibits, or halts the development of a tumor. An antineoplastic agent may be a targeted therapy drug (i.e., a drug that blocks the growth or spread of cancer by interfering with specific molecules that are involved in the growth, progression, or spread of cancer) or a traditional chemotherapeutic agent. Non-limiting examples of targeted therapies include hormone therapies, signal transduction inhibitors, gene expression modulators, apoptosis inducers, angiogenesis inhibitors, immunotherapies, and monoclonal antibodies that deliver toxic molecules. Additionally, numerous chemotherapeutic agents are used in the oncology art and include, for example, alkylating agents, antimetabolites, anthracyclines, plant alkaloids, and topoisomerase inhibitors. Examples of therapeutic agents administered for chemotherapy are well-known to the skilled artisan.

As used herein, the terms “blasts” and “blast cells” are used interchangeably to refer to undifferentiated, precursor blood stem cells. As used herein, the term “blast count” refers to the number of blast cells in a sample.

As used herein, an “effective amount” or “effective dose” refers to an amount of a compound that treats, upon single or multiple dose administration, a patient suffering from a condition. An effective amount can be determined by the attending diagnostician through the use of known techniques and by observing results obtained under analogous circumstances. In determining the effective amount, a number of factors are considered by the attending diagnostician, including, but not limited to: the patient's size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

In some embodiments, an effective dose is a dose that partially or fully alleviates (i.e., eliminates or reduces) at least one symptom associated with the disorder/disease state being treated, that slows, delays, or prevents onset or progression to a disorder/disease state, that slows, delays, or prevents progression of a disorder/disease state, that diminishes the extent of disease, that reverse one or more symptoms, that results in remission (partial or total) of disease, and/or that prolongs survival. Examples of disease states contemplated for treatment are set out herein. In some embodiments, the patient currently has cancer, was once treated for cancer and is in remission, or is at risk of relapsing after treatment for the cancer.

As used herein, the term “E-selectin antagonist” includes antagonists of E-selectin only, as well as antagonists of E-selectin and either P-selectin or L-selectin, and antagonists of E-selectin, P-selectin, and L-selectin. The terms “E-selectin antagonist” and “E-selectin inhibitor” are used interchangeably herein.

In some embodiments, the E-selectin antagonist inhibits an activity of E-selectin or inhibits the binding of E-selectin to one or more E-selectin ligands (which in turn may inhibit a biological activity of E-selectin).

E-selectin antagonists include the glycomimetic compounds described herein. E-selectin antagonists also include antibodies, polypeptides, peptides, peptidomimetics, and aptamers which bind at or near the binding site on E-selectin to inhibit E-selectin interaction with sialyl Lea (sLe^{a}) or sialyl Le^{x} (sLe^{x}).

Further disclosure regarding E-selectin antagonists suitable for the disclosed methods (e.g., compounds and compositions) may be found in U.S. Pat. No. 9,254,322, issued Feb. 9, 2016, and U.S. Pat. No. 9,486,497, issued Nov. 8, 2016, which are hereby incorporated by reference. In some embodiments, the E-selectin antagonist is chosen from E-selectin antagonists disclosed in U.S. Pat. No. 9,109,002, issued Aug. 18, 2015, which is hereby incorporated by reference. In some embodiments, the E-selectin antagonist is chosen from heterobifunctional antagonists disclosed in U.S. Pat. No. 8,410,066, issued Apr. 2, 2013, and U.S. Pat. No. 10,519,181, issued Dec. 31, 2019, which are hereby incorporated by reference. Further disclosure regarding E-selectin antagonists suitable for the disclosed methods and compounds may be found in U.S. Publication No. 2019/0233458, published Aug. 1, 2019, WO2019/133878, published Jul. 4, 2019, WO 2020/139962, published Jul. 2, 2020, WO 2020/219419, published Oct. 29, 2020, and WO 2020/219417, published Oct. 29, 2020, which are hereby incorporated by reference.

In some embodiments, the E-selectin antagonists suitable for the disclosed methods include pan-selectin antagonists. For example, heterobifunctional compounds for inhibition of E-selectin and the CXCR4 chemokine receptor comprising E-selectin inhibitor-Linker-CXCR4 chemokine receptor inhibitor are known in the art. Non-limiting examples are disclosed, for example, in U.S. Pat. No. 8,410,066.

As used herein, an amount expressed in terms of “mg of at least one compound chosen from [X] and pharmaceutically acceptable salts thereof” is based on the total weight of the free base of [X] present, in the form of the free base and/or one or more pharmaceutically acceptable salts of [X]. One of ordinary skill in the art would understand the amount of pharmaceutically acceptable derivative, such as a pharmaceutically acceptable salt, that is equivalent to the daily dosages and individual doses of a compound described herein. That is, for example, given the disclosure above of a fixed daily dose of 1600 mg of Compound A, one of ordinary skill in the art would understand how to determine an equivalent fixed daily dose of a pharmaceutically acceptable salt of Compound A.

As used herein, the term “increase” refers to altering positively by at least 1%, including, but not limited to, altering positively by at least 5% (e.g., by 5%), altering positively by at least 10% (e.g., 10%), altering positively by at least 25% (e.g., by 25%), altering positively by at least 30% (e.g., by 30%), altering positively by at least 50% (e.g., by 50%), altering positively by at least 75% (e.g., by 75%), or altering positively by 100%, altering positively by 5% to 10%, altering positively by 5% to 15%, altering positively by 5% to 25%, etc.

As used herein, the term “modulate” refers to altering positively or negatively. Non-limiting example modulations include an at least 1% (e.g., a 1%) change, an at least a 2% (e.g., 2%) change, an at least a 5% (e.g., 5%) change, an at least a 10% (e.g., a 10%) change, an at least a 25% (e.g., 25%) change, an at least a 50% (e.g., 50%) change, an at least a 75% (e.g., a 75%) change, a 100% change, a 5% to 10% change, a 5% to 15% change, a 5% to 25% change, etc.

As used herein, the terms “patient” and “subject” are used interchangeably. In some embodiments, the patient or subject is a mammal. In some embodiments, the patient or subject is a human.

As used herein, the term “pharmaceutical composition” refers to a mixture or a combination of at least one active pharmaceutical ingredient and at least one pharmaceutically acceptable excipient. Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail herein).

The pharmaceutical compositions described herein may be administered to a subject in need thereof by any of several routes that can effectively deliver an effective amount of the compound. In some embodiments, the pharmaceutical composition is administered parenterally. Non-limiting suitable routes of parenteral administration include subcutaneous, intravenous, intramuscular, intrasternal, intracavernous, intrameatal, and intraurethral injection and/or infusion. In some embodiments, the pharmaceutical composition is administered intravenously (IV). Non-limiting suitable routes of IV administration include via a peripheral line, a central catheter, and a peripherally inserted central line catheter (PICC). In some embodiments, the pharmaceutical composition is administered subcutaneously.

The pharmaceutical compositions described herein may be sterile aqueous or sterile non-aqueous solutions, suspensions, or emulsions, and may additionally comprise at least one pharmaceutically acceptable excipient or diluent (i.e., a non-toxic material that does not interfere with the activity of the active ingredient). Such compositions may be in the form of a solid, liquid, or gas (aerosol). A liquid pharmaceutical composition may include, for example, at least one the following: a sterile diluent such as water for injection; saline solution (e.g., physiological saline); Ringer's solution; isotonic sodium chloride; fixed oils that may serve as the solvent or suspending medium; polyethylene glycols; glycerin; propylene glycol or other solvents; antibacterial agents; antioxidants; chelating agents; buffers and agents for the adjustment of tonicity, such as, e.g., sodium chloride or dextrose. A parenteral preparation may be enclosed in ampoules, disposable syringes or multiple dose vials made of glass or plastic. In some embodiments, the pharmaceutical composition comprises physiological saline. In some embodiments, the pharmaceutical composition is an injectable pharmaceutical composition, and in some embodiments, the injectable pharmaceutical composition is sterile.

In some embodiments, a pharmaceutical composition is a solid pharmaceutical composition. In some embodiments, a pharmaceutical composition is a pharmaceutical composition for oral administration. In some embodiments, a pharmaceutical composition is a single dosage unit form. In some embodiments, a pharmaceutical composition is a multiple dosage unit form. In some embodiments, a pharmaceutical composition is a tablet composition. In some embodiments, a pharmaceutical composition is a capsule composition.

In some embodiments, a pharmaceutical composition is formulated as a liquid. In some embodiments, a pharmaceutical composition is formulated as a liquid for intravenous administration. In some embodiments, a pharmaceutical composition is formulated as a liquid for parenteral administration. In some embodiments, a pharmaceutical composition is formulated as a liquid for subcutaneous (subQ) administration. In some embodiments, a pharmaceutical composition is formulated as a liquid for intramuscular (IM) administration. In some embodiments, a pharmaceutical composition is formulated as a liquid for intraosseous administration.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which in the aggregate can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

In general, the type of excipient or diluent is selected based on the mode of administration, as well as the chemical composition of the active ingredient(s). As a non-limiting example, pharmaceutical compositions for parenteral administration may further comprise one or more of water, saline, alcohols, fats, waxes, and buffers.

As used herein, the term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

As used herein, the term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

As used herein, the term “reduce” refers to altering negatively by at least 1% including, but not limited to, altering negatively by at least 5% (e.g., by 5%), altering negatively by at least 10% (e.g., by 10%), altering negatively by at least 25% (e.g., by 25%), altering negatively by at least 30% (e.g., by 30%), altering negatively by at least 50% (e.g., by 50%), altering negatively by at least 75% (e.g., by 75%), altering negatively by 100%, altering negatively by 5% to 10%, altering negatively by 5% to 15%, altering negatively by 5% to 25%, etc.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. The effect can be prophylactic in terms of completely or partially preventing a disease or symptom thereof from occurring in the first place and/or can be therapeutic in terms of a partial or complete cure for a disease and/or adverse effects attributable to the disease. As a non-limiting example, the term “treatment” and the like, as used herein, encompasses any treatment of cancers, such as, e.g., AML or any of its subtypes and related hematologic cancers in a mammal, such as, e.g., in a human, and includes: (a) preventing the disease from occurring in a subject, e.g., a subject identified as predisposed to the disease or at risk of acquiring the disease but has not yet been diagnosed as having it; (b) delaying onset or progression of the disease, e.g., as compared to the anticipated onset or progression of the disease in the absence of treatment; (c) inhibiting the disease, i.e., arresting its development; and/or (d) relieving the disease, i.e., causing regression of the disease. Improvements in or lessening the severity of any symptom of the disorder or condition can be readily assessed according to standard methods and techniques known in the art.

In some embodiments, “treating” refers to administering, e.g., subcutaneously, an effective dose or effective multiple doses of a composition, e.g., a composition comprising at least one E-selectin antagonist as disclosed herein, to an animal (including a human being) suspected of suffering or already suffering from AML or another related cancer.

In some embodiments, “treating” can also refer to reducing, eliminating, or at least partially arresting, as well as to exerting any beneficial effect, on one or more symptoms of the disease and/or associated with the disease and/or its complications.

### Non-Limiting Example Embodiments 1

Without limitation, some example embodiments of the present disclosure include:

1. A method of treating acute myeloid leukemia (AML) in a subject in need thereof comprising administering to the subject at least one E-selectin inhibitor in combination with venetoclax and at least one hypomethylating agent.
2. The method of Embodiment 1, wherein the at least one E-selectin inhibitor is chosen from carbohydrate mimetics of an E-selectin ligand.
3. The method of Embodiment 1 or 2, wherein the at least one E-selectin inhibitor is chosen from

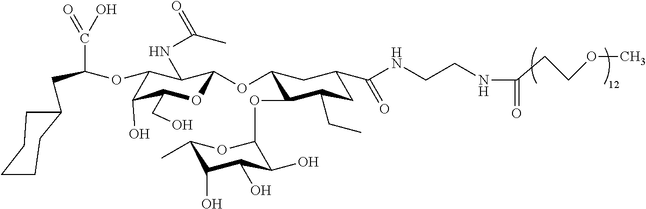

and pharmaceutically acceptable salts thereof.
4. The method of any one of Embodiments 1-3, wherein the at least one hypomethylating agent is 5-azacitidine.
5. The method of any one of Embodiments 1-4, wherein the subject has acquired resistance to a combination therapy comprising venetoclax and at least one hypomethylating agent.

### Non-Limiting Example Embodiments 2

Without limitation, some example embodiments/clauses of the present disclosure include:

1. A method of treating a cancer in a subject in need thereof comprising administering to the subject at least one E-selectin antagonist, wherein the subject is further administered venetoclax.
2. A method of treating a cancer in a subject in need thereof comprising administering to the subject at least one E-selectin antagonist, wherein the subject is further administered at least one hypomethylating agent.
3. A method of treating a cancer in a subject in need thereof comprising administering to the subject at least one E-selectin antagonist, wherein the subject is further administered at least one antineoplastic agent and at least one hypomethylating agent.
4. The method according to Clause 2 or 3, wherein the at least one hypomethylating agent is chosen from 5-azacitidine, decitabine, guadecitabine, 5-fluoro-2′-deoxycytidine, zebularine, CP-4200, RG108, and nanaomycin A.
5. The method according to any one of Clauses 2-4, wherein the at least one hypomethylating agent is 5-azacitidine.
6. The method according to any one of Clauses 2-4, wherein the at least one hypomethylating agent is decitabine.
7. The method according to any one of Clauses 3-6, wherein the at least one antineoplastic agent is chosen from targeted therapy drugs.
8. The method according to any one of Clauses 3-7, wherein the at least one antineoplastic agent is venetoclax.
9. The method according to any one Clauses 1-8, wherein the method comprises administering to the subject a fixed dose of 10 mg to 1000 mg (such as, e.g., 10 mg, 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, e.g., 20 mg to 400 mg) per day of venetoclax.
the method comprises administering to the subject a fixed dose of 400 mg per day of venetoclax.
10. The method according to any one of Clauses 3-6, wherein the at least one antineoplastic agent is chosen from chemotherapeutic agents.
11. The method according to any one of Clauses 1-10, wherein the at least one E-selectin antagonist is chosen from carbohydrate mimetics of an E-selectin ligand.
12. The method according to any one of Clauses 1-11, wherein the at least E-selectin antagonist is chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing.
13. The method according to any one of Clauses 1-12, wherein the at least E-selectin antagonist is chosen from Compound A, Compound B, Compound C, Compound D, Compound E, and pharmaceutically acceptable salts of any of the foregoing.
14. The method according to any one of Clauses 1-13, wherein the at least one E-selectin antagonist is chosen from

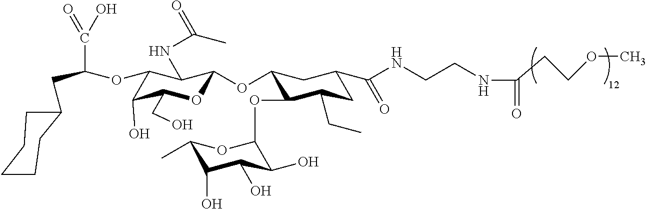

and pharmaceutically acceptable salts thereof.
15. The method according to any one of Clauses 1-14, wherein the method comprises administering to the subject a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg, 1000 mg to 2000 mg) per day of the at least one E-selectin antagonist.
16. The method according to any one of Clauses 1-14, wherein the method comprises administering to the subject a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist.
17. The method according to any one of Clauses 1-16, wherein the cancer is chosen from liquid cancers.
18. The method according to any one of Clauses 1-16, wherein the cancer is chosen from solid cancers.
19. The method according to any one of Clauses 1-18, wherein the cancer is chosen from FLT3 mutated cancers.
20. The method according to any one of Clauses 1-19, wherein the cancer is chosen from FLT3-ITD mutated cancers.
21. The method according to any one of Clauses 1-20, wherein the cancer is chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.
22. The method according to any one of Clauses 1-21, wherein the cancer is chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.
23. The method according to Clause 21 or 22, wherein the leukemia is chosen from acute myeloid leukemia, acute lymphocytic leukemia, chronic lymphocytic leukemia, and chronic myelogenous leukemia.
24. The method according to any one of Clauses 1-17 and 19-23, wherein the cancer is AML.
25. The method according to any one of Clauses 1-17 and 19-24, wherein the cancer is relapsed/refractory AML.
26. The method according to any one of Clauses 1-17 and 19-25, wherein the cancer is FLT3-ITD mutated AML.
27. The method according to Clause 21 or 22, wherein the lymphoma is chosen from non-Hodgkin's lymphoma and Hodgkin's lymphoma.
28. The method according to Clause 21 or 22, wherein the myeloma is multiple myeloma.
29. The method according to Clause 21 or 22, wherein the melanoma is chosen from uveal melanoma and skin melanoma.
30. The method according to any one of Clauses 1-29, wherein the subject has acquired resistance to a therapy comprising at least one antineoplastic agent.
31. The method according to any one of Clauses 1-30, wherein the subject has acquired resistance to a therapy comprising venetoclax.
32. The method according to any one of Clauses 1-31, wherein the subject has acquired resistance to a therapy comprising sorafenib.
33. The method according to any one of Clauses 1-32, wherein the subject has acquired resistance to a therapy comprising at least one hypomethylating agent.
34. The method according to any one of Clauses 1-33, wherein the subject has acquired resistance to a combination therapy comprising at least one antineoplastic agent and at least one hypomethylating agent.
35. The method according to any one of Clauses 1-34, wherein the subject has acquired resistance to a combination therapy comprising venetoclax and at least one hypomethylating agent.
36. The method according to any one of Clauses 1-35, wherein the subject possesses one or more mutational alterations of FLT3.
37. The method according to Clause 36, wherein the mutational alterations are chosen from internal tandem duplications and missense mutations within the tyrosine kinase domain activation loop of FLT3.
38. The method according to Clause 36 or 37, wherein the mutational alterations are chosen from internal tandem duplications within the tyrosine kinase domain activation loop of FLT3.
39. The method according to Clause 36 or 37, wherein the mutational alterations are chosen from missense mutations within the tyrosine kinase domain activation loop of FLT3.
40. The method according to any one of Clauses 1-39, wherein the subject expresses the gene ST3GAL4 at an expression level greater than that of at least 55% (such as, e.g., 55%, 60%, 65%, 70%, 75%, 80%, 85%, 90%, or 95%) of cancer patients.
41. The method according to any one of Clauses 1-40, wherein the subject expresses the gene B3GNT5 at an expression level greater than that of at least 55% (such as, e.g., 55%, 60%, 65%, 70%, 75%, 80%, 85%, 90%, or 95%) of cancer patients.
42. The method according to any one of Clauses 1-41, wherein the subject expresses the gene FUT7 at an expression level greater than that of at least 55% (such as, e.g., 55%, 60%, 65%, 70%, 75%, 80%, 85%, 90%, or 95%) of cancer patients.
43. The method according to any one of Clauses 1-42, wherein the method further comprises selecting the subject to treat through a method comprising: (a) determining or having determined the gene expression level of one or more genes in the subject or a sample from the subject; and (b) selecting the subject for treatment when at least 10% of the blast cells from the subject or sample from the subject expresses the one or more genes.
44. The method according to Clause 43, wherein the gene expression level is measured by the amount of mRNA.
45. The method according to Clause 43, wherein the gene expression level is measured by the amount of protein in the sample from the subject.
46. The method according to any one of Clauses 43-45, wherein the sample from the subject is peripheral blood.
47. The method according to any one of Clauses 43-46, wherein the one or more genes are chosen from ST3GAL4, B3GNT5, and FUT7.
48. The method according to any one of Clauses 1-42, wherein the method further comprises selecting the subject to treat through a method comprising: (a) obtaining or having obtained a biological sample comprising blast cells from the subject; (b) performing or having performed an assay on the biological sample to determine the gene expression level of one or more E-selectin ligand-forming genes in the sample; and (c) selecting the subject for treatment when at least 10% of the blast cells in the sample express the one or more E-selectin ligand-forming genes.
49. The method according to Clause 48, wherein the biological sample is a bone marrow sample.
50. The method according to Clause 48, wherein the biological sample is a peripheral blood sample.
51. The method according to any one of Clauses 48-50, wherein the one or more E-selectin ligand-forming genes are glycosylation genes.
52. The method according to any one of Clauses 48-51, wherein the one or more E-selectin-ligand forming genes are chosen from ST3GAL4 and FUT7.
53. The method according to any one of Clauses 1-42, wherein the method further comprises selecting the subject to treat through a method comprising: (a) determining the gene expression level of one or more genes in the subject or a sample from the subject; (b) comparing the gene expression level from (a) to a control sample from a cancer-free subject, a newly diagnosed cancer subject, or a subject diagnosed with the same cancer as the subject, and (c) selecting the subject for treatment when the gene expression level exceeds that in the control sample.
54. The method according to Clause 53, wherein the gene expression level is measured by the amount of mRNA.
55. The method according to Clause 53, wherein the gene expression level is measured by the amount of protein in the sample from the subject.
56. The method according to any one of Clauses 53-55, wherein the one or more genes are chosen from ST3GAL4, B3GNT5, and FUT7.
57. The method according to any one of Clauses 1-56, wherein the subject is receiving, has received, or will receive two or more chemotherapeutic agents (such as, e.g., mitoxantrone, etoposide, and cytarabine or fludarabine, cytarabine, and idarubicin).
58. The method according to any one of Clauses 1-57, wherein the subject is receiving, has received, or will receive velafermin, palifermin, thalidomide, and/or a thalidomide derivative.
59. The method according to any one of Clauses 1-58, wherein the subject is receiving, has received, or will receive MMP inhibitors, inflammatory cytokine inhibitors, mast cell inhibitors, NSAIDs, NO inhibitors, MDM2 inhibitors, or antimicrobial compounds.
60. The method according to any one of Clauses 1-59, wherein the administration extends the number of days the subject is in remission, reduces the number of days until remission, inhibits the metastasis of cancer cells, or improves survival.
61. The method according to any one of Clauses 1-60, wherein the subject is a human.

Some embodiments of the present disclosure relate to a method of treating a cancer in a subject in need thereof comprising administering to the subject at least one E-selectin antagonist, wherein the subject is further administered at least one antineoplastic agent and/or at least one hypomethylating agent. In some embodiments, the at least one E-selectin antagonist is chosen from carbohydrate mimetics of an E-selectin ligand.

In some embodiments, the at least one E-selectin antagonist is chosen from Compound A and pharmaceutically acceptable salts thereof.

In some embodiments, the at least one E-selectin antagonist is Compound A.

In some embodiments, the at least one E-selectin antagonist is chosen from compounds of Formula (I):

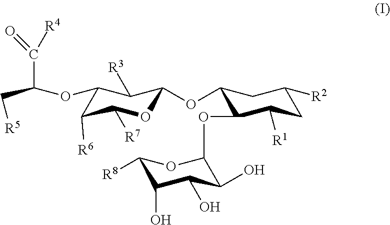

isomers of Formula (I), tautomers of Formula (I), and pharmaceutically acceptable salts of any of the foregoing, wherein:
R¹ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from H, -M, and -L-M;
R³ is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R⁴ is chosen from —OH and —NZ¹Z² groups, wherein Z¹ and Z², which may be identical or different, are each independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY groups, wherein Y is chosen from C₁₋₄ alkyl, C₂₋₄ alkenyl, and C₂₋₄ alkynyl groups.

In some embodiments, the at least one E-selectin antagonist is chosen from compounds of Formula (I), wherein the non-glycomimetic moiety comprises polyethylene glycol.

In some embodiments, the at least one E-selectin antagonist is chosen from compounds of Formula (I), wherein L is —C(═O)NH(CH₂)₁₋₄NHC(═O)— and the non-glycomimetic moiety comprises polyethylene glycol.

In some embodiments, the at least one E-selectin antagonist is chosen from compounds of Formula (Ia):

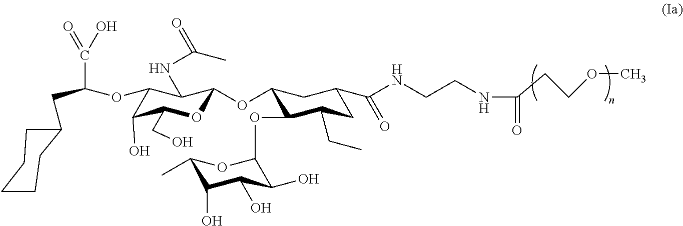

and pharmaceutically acceptable salts thereof, wherein n is chosen from integers ranging from 1 to 100. In some embodiments, n is chosen from 4, 8, 12, 16, 20, 24, and 28. In some embodiments n is 12.

In some embodiments, the at least one E-selectin antagonist is chosen from Compound A:

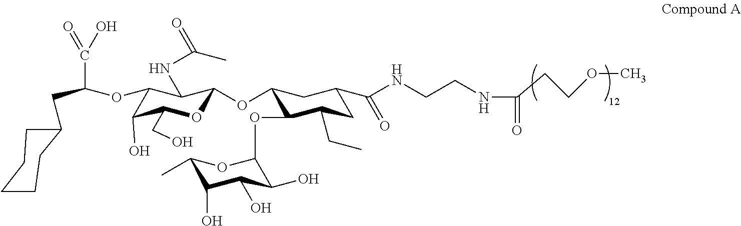

and pharmaceutically acceptable salts thereof.

In some embodiments, the at least one E-selectin antagonist is a heterobifunctional inhibitor of E-selectin and CXCR4 chosen from compounds of Formula (II):

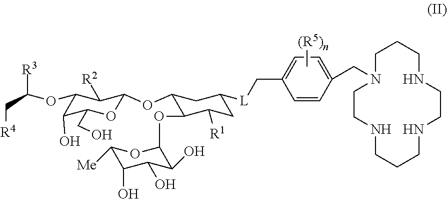

isomers of Formula (II), tautomers of Formula (II), and pharmaceutically acceptable salts of any of the foregoing, wherein:
R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R² is chosen from —OH, —NH₂, —OC(═O)Y¹, —NHC(═O)Y¹, and —NHC(═O)NHY¹ groups, wherein Y¹ is chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups;
R³ is chosen from —CN, —CH₂CN, and —C(═O)Y² groups, wherein Y² is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Z¹ and Z² may together form a ring;
R⁴ is chosen from C₃₋₈ cycloalkyl groups;
R⁵ is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
n is chosen from integers ranging from 1 to 4; and
L is chosen from linker groups.

In some embodiments, the at least one E-selectin antagonist is chosen from compounds of Formula (IIa):

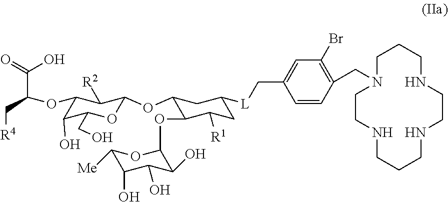

and pharmaceutically acceptable salts thereof.

In some embodiments, the at least one E-selectin antagonist is chosen from Compound B:

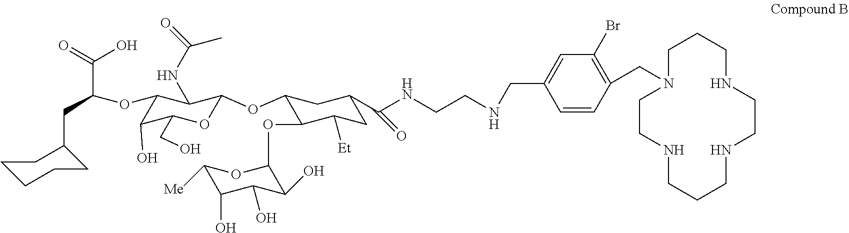 [SMILES: CCC1CC(C(=O)NCCNCc2ccc(CN3CCCNCCNCCCNCC3)c(Br)c2)CC(OC2OC(CO)C(O)C(O[C@@H](CC3CCCCC3)C(=O)O)C2NC(C)=O)C1OC1OC(C)C(O)C(O)C1O]

and pharmaceutically acceptable salts thereof.

In some embodiments, the at least one E-selectin antagonist is a heterobifunctional pan-selectin antagonist chosen from compounds of Formula (III):

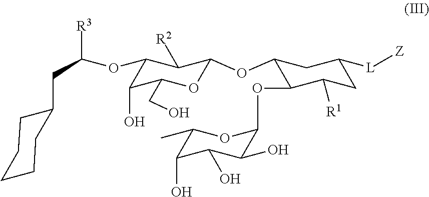

isomers of Formula (III), tautomers of Formula (III), and pharmaceutically acceptable salts of any of the foregoing, wherein:
R¹ is chosen from H, C₁₋₈ alkyl, C₂₋₈ alkenyl, C₂₋₈ alkynyl, C₄₋₁₆ cycloalkylalkyl

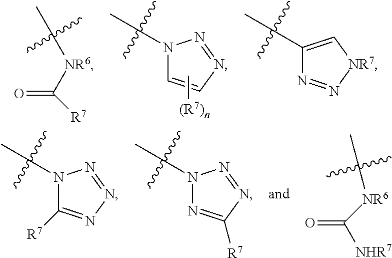

groups;
R² is chosen from C₁₋₈ alkyl, C₂₋₈ alkenyl, C₂₋₈ alkynyl, C₄₋₁₆ cycloalkylalkyl, —OH, —OX¹, halo, —NH₂, —OC(═O)X¹, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups;
R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring;
R⁶ is chosen from H, C₁₋₈ alkyl, C₂₋₈ alkenyl, C₂₋₈ alkynyl, C₄₋₁₆ cycloalkylalkyl, and —C(═O)R⁷ groups;
each R⁷ is independently chosen from H, C₁₋₈ alkyl, C₂₋₈ alkenyl, C₂₋₈ alkynyl, C₄₋₁₆ cycloalkylalkyl,

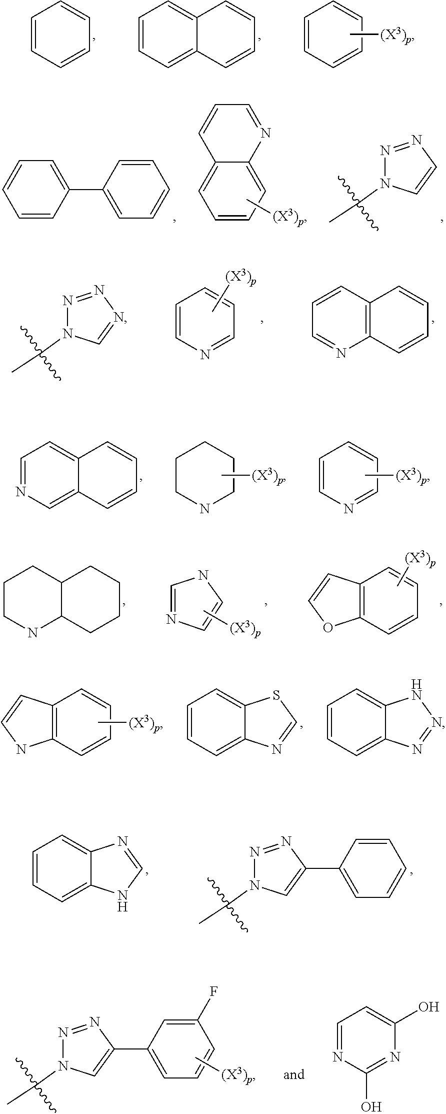

groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;
n is chosen from integers ranging from 0 to 2;
p is chosen from integers ranging from 0 to 3;
L is chosen from linker groups; and
Z is chosen from benzyl amino sulfonic acid groups.

Benzyl amino sulfonic acids (BASAs) are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion.

Further disclosure regarding BASAs suitable for the disclosed compounds may be found in U.S. Reissue Patent No. RE44,778, issued Feb. 25, 2014, and U.S. Publication No. US2018/0369205, published Dec. 27, 2018, which are hereby incorporated by reference in their entireties.

In some embodiments, the at least one E-selectin antagonist is a heterobifunctional pan-selectin antagonist chosen from compounds of Formula (IIIa):

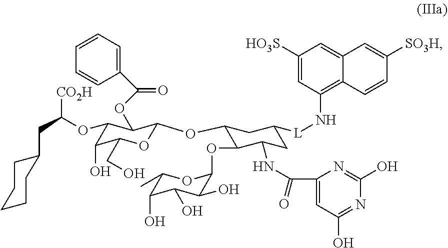

tautomers of Formula (IIIa), and pharmaceutically acceptable salts of any of the foregoing.

In some embodiments, the at least one E-selectin antagonist is a heterobifunctional pan-selectin antagonist chosen from Compound C:

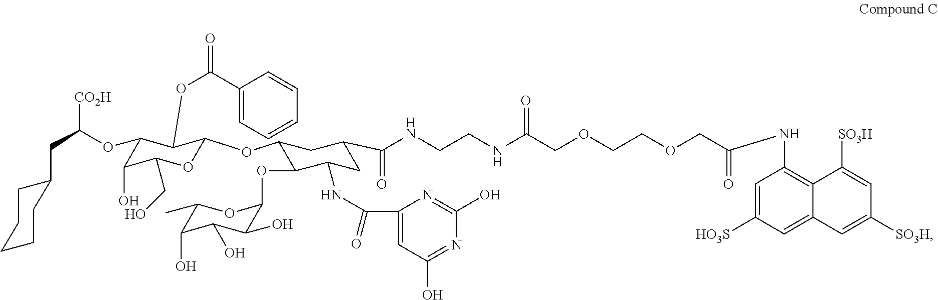 [SMILES: CC1OC(OC2C(NC(=O)c3cc(O)nc(O)n3)CC(C(=O)NCCNC(=O)COCCOCC(=O)Nc3cc(S(=O)(=O)O)cc4cc(S(=O)(=O)O)cc(S(=O)(=O)O)c34)CC2OC2OC(CO)C(O)C(O[C@@H](CC3CCCCC3)C(=O)O)C2OC(=O)c2ccccc2)C(O)C(O)C1O]

tautomers of Compound C, and pharmaceutically acceptable salts of any of the foregoing.

In some embodiments, the linker groups of Formula (I), Formula (II), and/or Formula (III) are independently chosen from groups comprising spacer groups, such spacer groups as, for example, —(CH₂)ₚ— and —O(CH₂)ₚ—, wherein p is chosen from integers ranging from 1 to 30. In some embodiments, p is chosen from integers ranging from 1 to 20.

Other non-limiting examples of spacer groups include carbonyl groups and carbonyl-containing groups such as, for example, amide groups.

In some embodiments, the linker group of Formula (I), Formula (II), and/or Formula (III) is chosen from

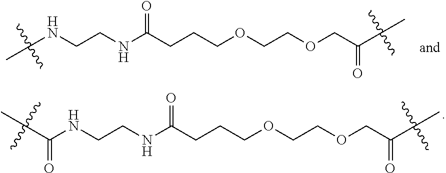 [SMILES: *NCCNC(=O)CCCOCCOCC(*)=O ; *C(=O)COCCOCCCC(=O)NCCNC(*)=O]

In some embodiments, the linker group of Formula (I), Formula (II), and/or Formula (III) is chosen from

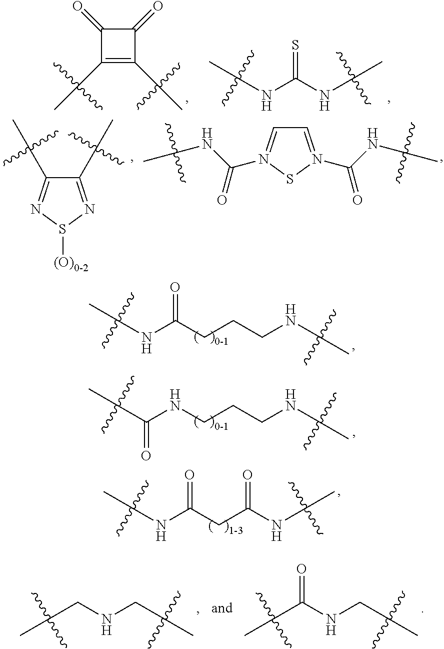

Other linker groups, such as, for example, polyethylene glycols (PEGs) and —C(═O)—NH—(CH₂)ₚ—C(═O)—NH—, wherein p is chosen from integers ranging from 1 to 30, or wherein p is chosen from integers ranging from 1 to 20, will be familiar to those of ordinary skill in the art and/or those in possession of the present disclosure.

In some embodiments, the linker group of Formula (I), Formula (II), and/or Formula (III) is chosen from

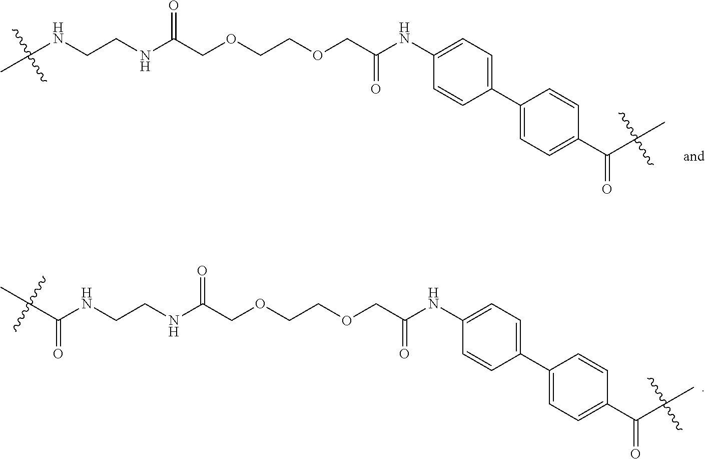 [SMILES: *NCCNC(=O)COCCOCC(=O)Nc1ccc(-c2ccc(C(*)=O)cc2)cc1 ; *C(=O)NCCNC(=O)COCCOCC(=O)Nc1ccc(-c2ccc(C(*)=O)cc2)cc1]

In some embodiments, the linker group of Formula (I), Formula (II), and/or Formula (III) is chosen from

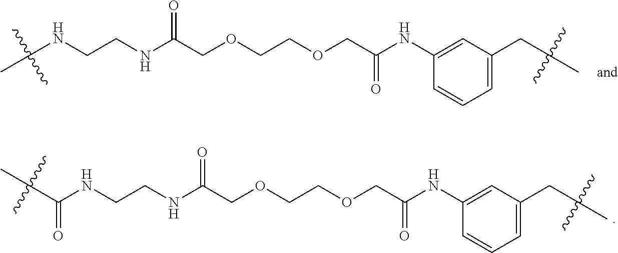 [SMILES: *Cc1cccc(NC(=O)COCCOCC(=O)NCCN*)c1 ; *Cc1cccc(NC(=O)COCCOCC(=O)NCCNC(*)=O)c1]

In some embodiments, the linker group of Formula (I), Formula (II), and/or Formula (III) is chosen from

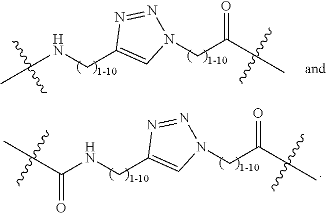

In some embodiments, the linker group of Formula (I), Formula (II), and/or Formula (III) is chosen from —C(═O)NH(CH₂)₂NH—, —CH₂NHCH₂—, and —C(═O)NHCH₂—. In some embodiments, the linker group is —C(═O)NH(CH₂)₂NH—.

In some embodiments, the at least one E-selectin antagonist is chosen from compounds of Formula (IV):

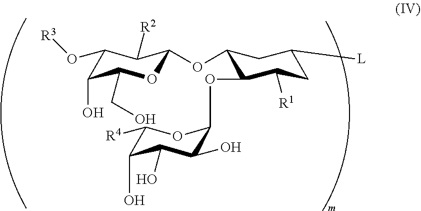

prodrugs of Formula (IV), isomers of Formula (IV), tautomers of Formula (IV), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

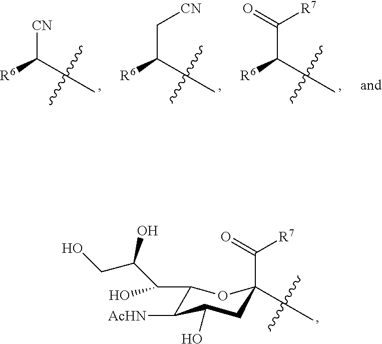

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

In some embodiments, the at least one E-selectin antagonist is chosen from compounds of Formula (V):

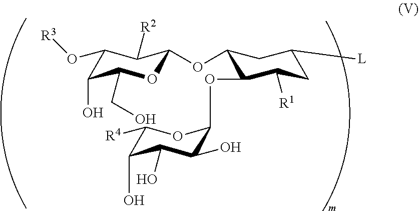

prodrugs of Formula (V), isomers of Formula (V), tautomers of Formula (V), and pharmaceutically acceptable salts of any of the foregoing, wherein:
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NYiY² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from

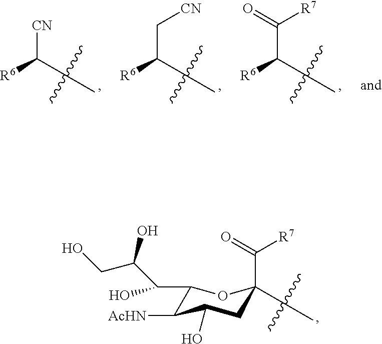

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is 2; and
L is chosen from

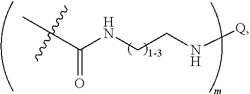

wherein Q is a chosen from

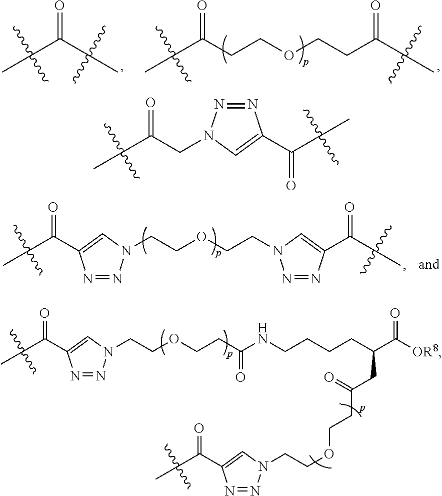

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

In some embodiments, the at least one E-selectin antagonist of Formula (IV) or Formula (V) is chosen from compounds of the following Formula (IVa/Va) (see definitions of L and m for Formula (IV) or (V) above):

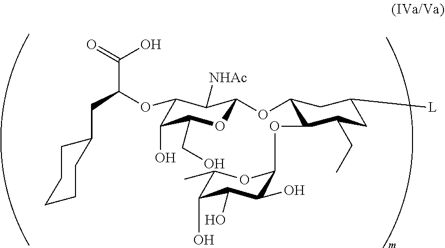

In some embodiments, the at least one E-selectin antagonist of Formula (IV) or Formula (V) is chosen from compounds of the following Formula (IVb/Vb) (see definitions of L and m for Formula (IV) or (V) above):

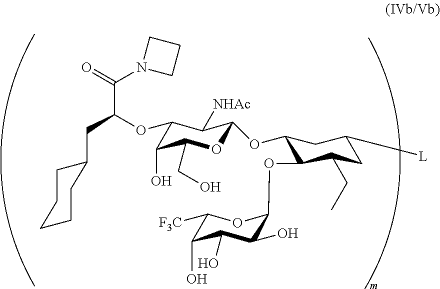

In some embodiments, the at least one E-selectin antagonist is Compound D:

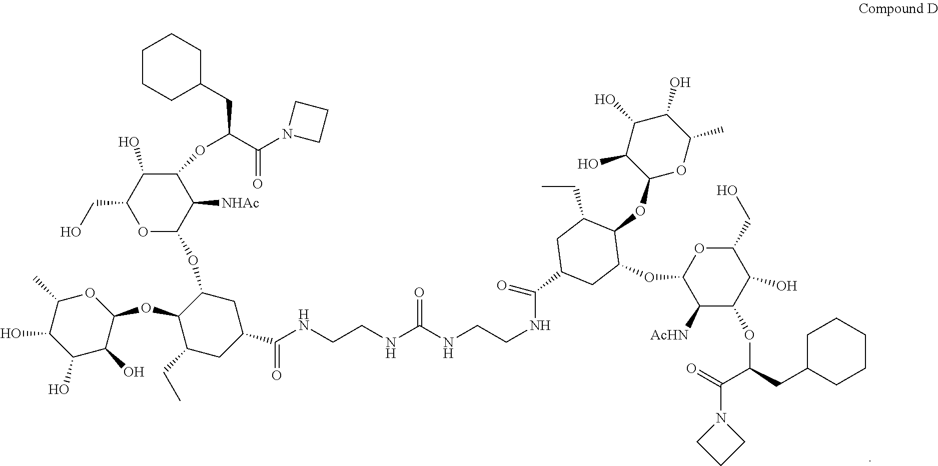

In some embodiments, the at least one E-selectin inhibitor is a heterobifunctional inhibitor of E-selectin and galectin-3, chosen from compounds of Formula (VI):

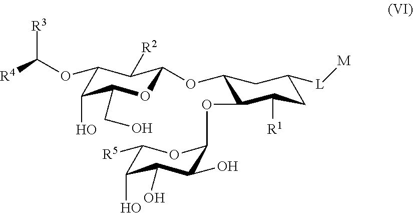

prodrugs of Formula (VI), isomers of Formula (VI), tautomers of Formula (VI), and pharmaceutically acceptable salts of any of the foregoing, wherein
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

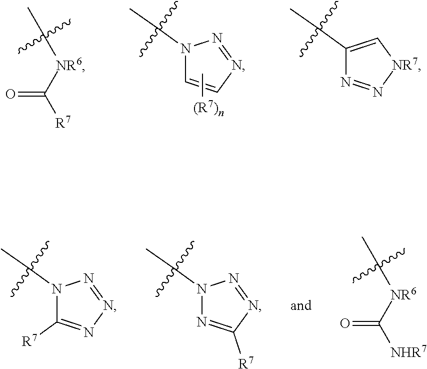

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, wherein Y¹ and Y² may join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, wherein Z¹ and Z² may join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

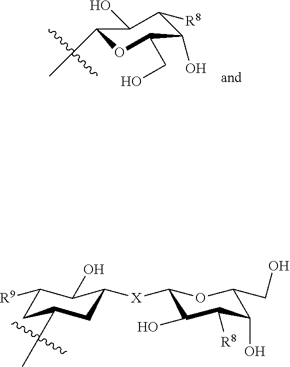

groups, wherein X is chosen from O and S, and R⁸ and R⁹, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y⁴ groups, wherein Y⁴ is chosen from C₁₋₈ alkyl, C₂₋₁₂ heterocyclyl, C₆₋₁₈ aryl, and C₁₋₁₃ heteroaryl groups; and
L is chosen from linker groups.

In some embodiments, the at least one E-selectin antagonist is chosen from compounds having the following Formulae:

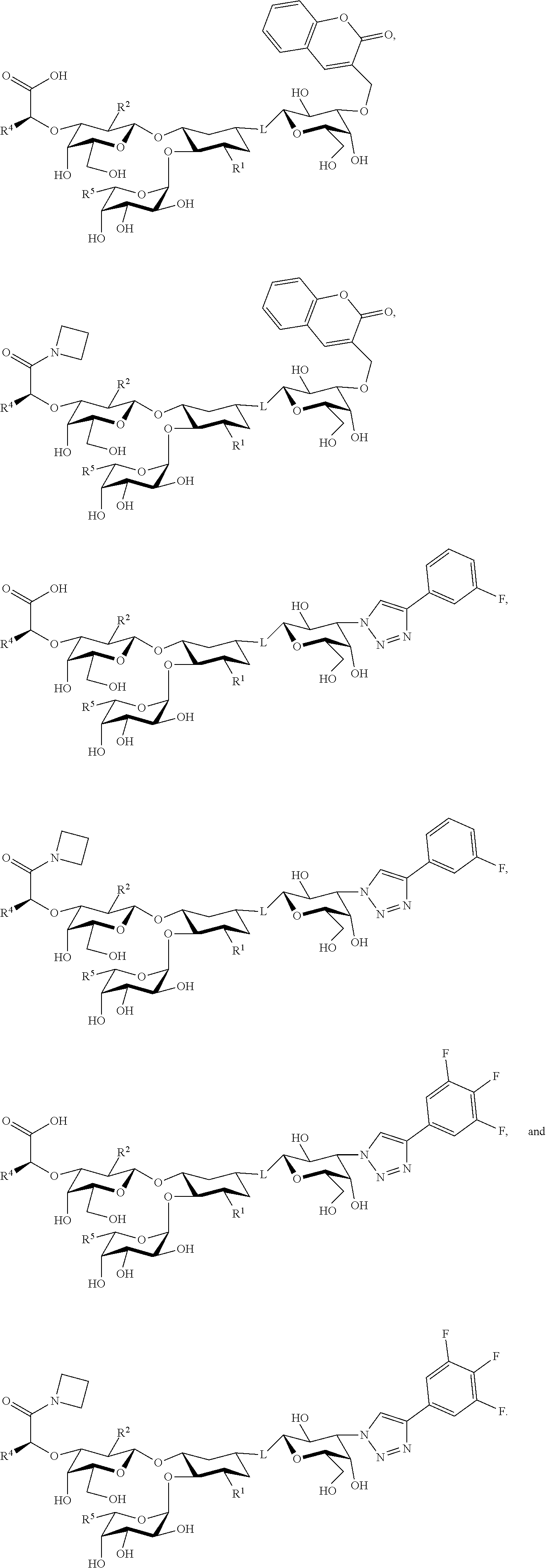

In some embodiments, the at least one E-selectin antagonist is chosen from compounds having the following Formulae:

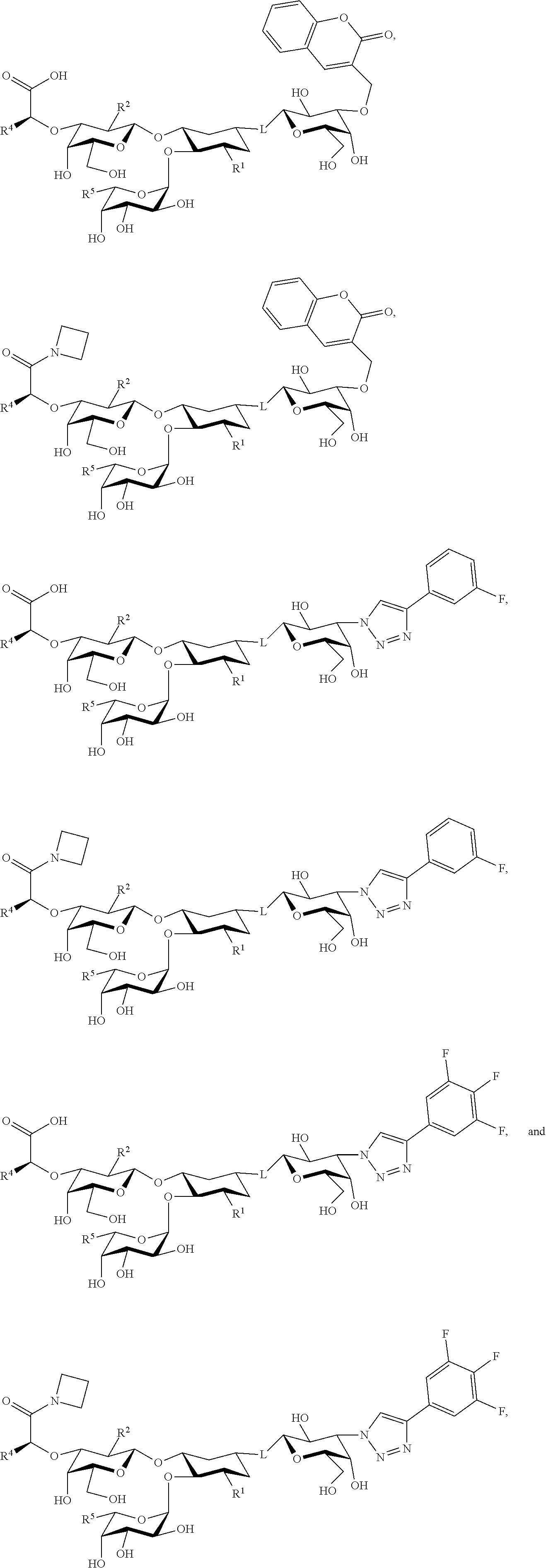

and pharmaceutically acceptable salts of any of the foregoing.

In some embodiments, the at least one E-selectin antagonist is chosen from compounds having the following Formulae:

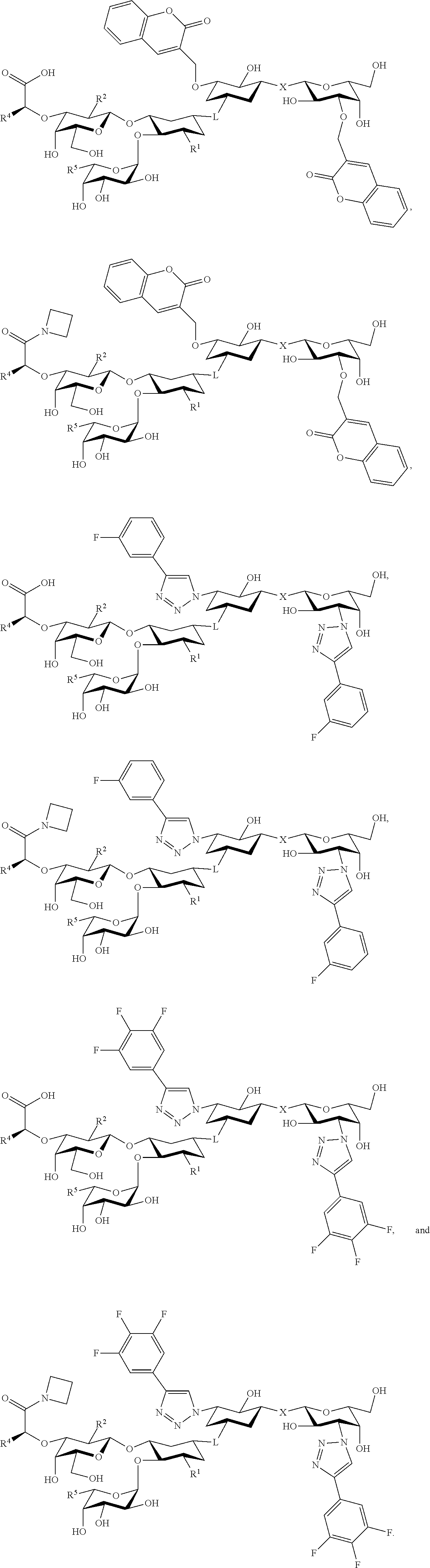

In some embodiments, the at least one E-selectin antagonist is chosen from compounds having the following Formulae:

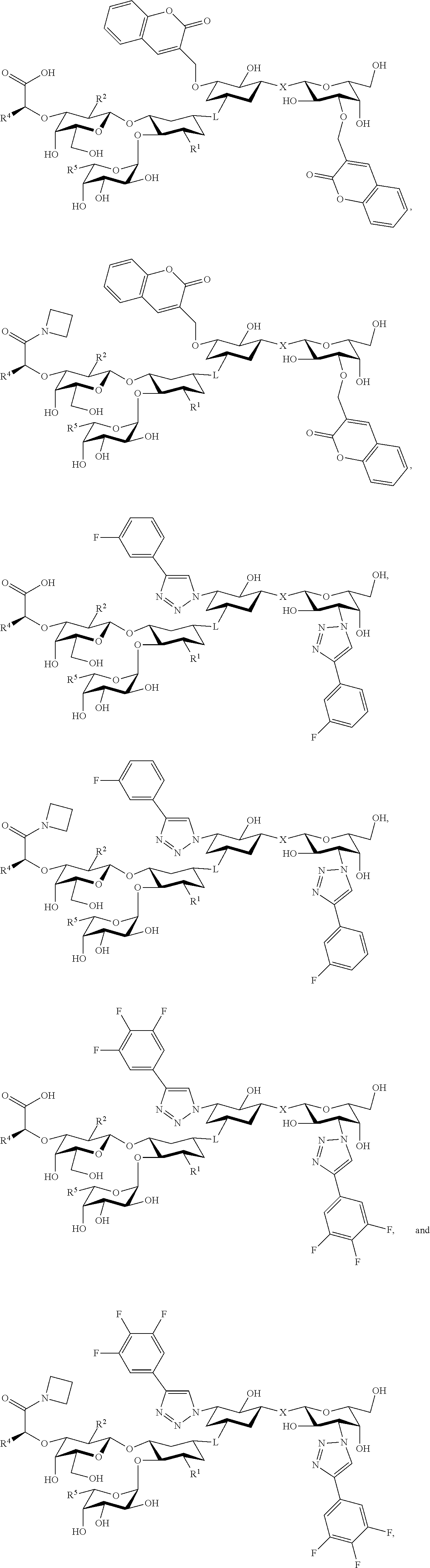

and pharmaceutically acceptable salts of any of the foregoing.

In some embodiments, the at least one E-selectin antagonist is Compound E:

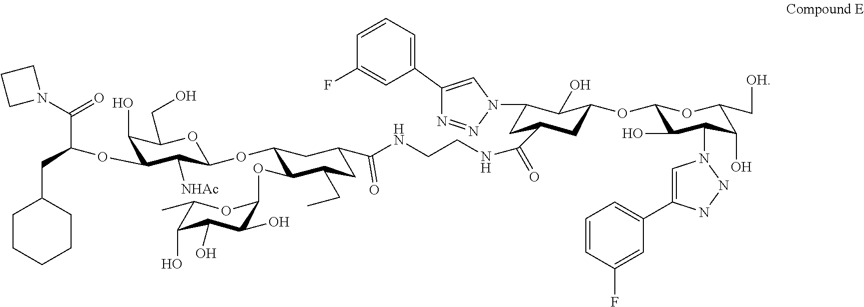

In some embodiments, the at least one E-selectin antagonist is chosen from compounds of Formula (VII):

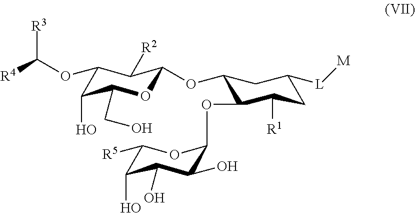

prodrugs of Formula (VII), isomers of Formula (VII), tautomers of Formula (VII), and pharmaceutically acceptable salts of any of the foregoing, wherein
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

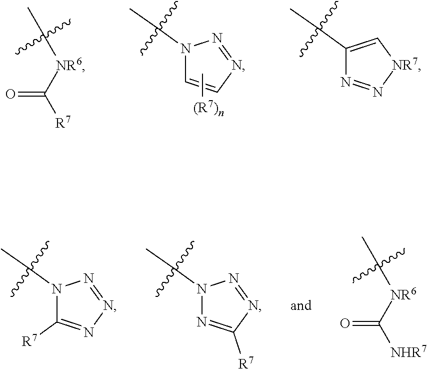

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, or Y¹ and Y² join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇-12 arylalkyl groups, or Z¹ and Z² join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

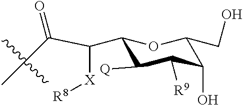

groups,
wherein
   X is chosen from —O—, —S—, —C—, and —N(R¹⁰)—, wherein R¹⁰ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups,
   Q is chosen from H, halo, and —OZ³ groups, wherein Z³ is chosen from H and C₁₋₈ alkyl groups,
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z⁵, which may be identical or different, are independently chosen from H, C₁₋₈ alkyl, and C₁₋₈ haloalkyl groups, or Z⁴ and Z⁵ join together along with the nitrogen atom to which they are attached to form a ring,
R⁹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹¹, C₁₋₈ alkyl, C₁₋₈ haloalkyl, —C(═O)OZ⁶, and —C(═O)NZ⁶Z⁷ groups, wherein R¹¹ is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein Z⁶, Z⁷, Z⁸ and Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring, and
wherein each of Z³, Z⁴, Z⁵, Z⁶, Z⁷, Z⁸, and Z⁹ is optionally substituted with one or more groups independently chosen from halo and —OR¹² groups, wherein R¹² is independently chosen from H and C₁₋₈ alkyl groups; and
L is chosen from linker groups.

In some embodiments of Formula (VII), M is chosen from

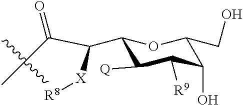

groups.

In some embodiments of Formula (VII), M is chosen from

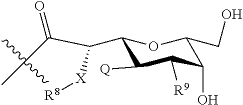

groups.

In some embodiments of Formula (VII), linker groups may be chosen from groups comprising spacer groups, such spacer groups as, for example, —(CH₂)ₜ— and —O(CH₂)ₜ—, wherein t is chosen from integers ranging from 1 to 20. Other non-limiting examples of spacer groups include carbonyl groups and carbonyl-containing groups such as, for example, amide groups. A non-limiting example of a spacer group is

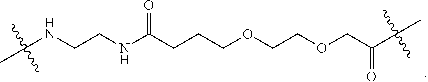 [SMILES: *NCCNC(=O)CCCOCCOCC(*)=O]

In some embodiments of Formula (VII), the linker group is chosen from

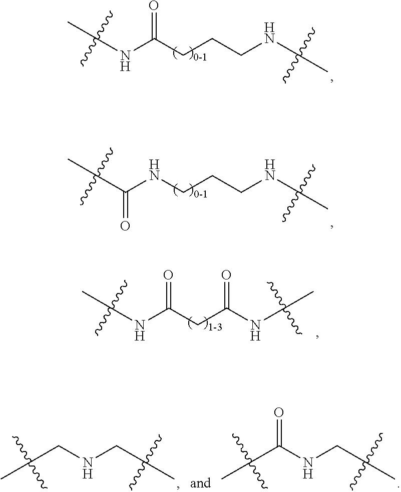

In some embodiments of Formula (VII), the linker group is chosen from polyethylene glycols (PEGs), —C(═O)NH(CH₂)ᵥO—, —C(═O)NH(CH₂)ᵥNHC(═O), —C(═O)NHC(═O)(CH₂)NH—, and —C(═O)NH(CH₂)ᵥC(═O)NH— groups, wherein v is chosen from integers ranging from 2 to 20. In some embodiments, v is chosen from integers ranging from 2 to 4. In some embodiments, v is 2. In some embodiments, v is 3. In some embodiments, v is 4.

In some embodiments of Formula (VII), the linker group is

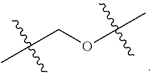 [SMILES: *CO*]

In some embodiments of Formula (VII), the linker group is

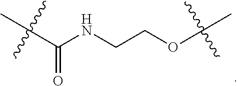 [SMILES: *OCCNC(*)=O]

In some embodiments of Formula (VII), the linker group is

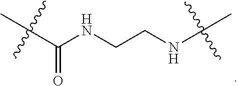 [SMILES: *NCCNC(*)=O]

In some embodiments of Formula (VII), the linker group is

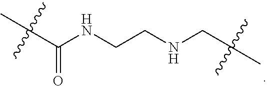 [SMILES: *CNCCNC(*)=O]

In some embodiments of Formula (VII), the linker group is

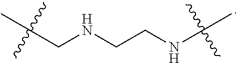 [SMILES: *CNCCN*]

In some embodiments of Formula (VII), the linker group is

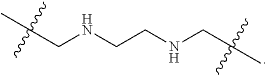 [SMILES: *CNCCNC*]

In some embodiments of Formula (VII), the linker group is

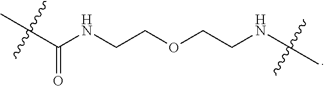 [SMILES: *NCCOCCNC(*)=O]

In some embodiments of Formula (VII), the linker group is

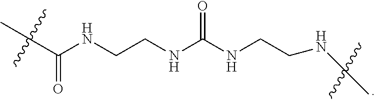 [SMILES: *NCCNC(=O)NCCNC(*)=O]

In some embodiments of Formula (VII), the linker group is

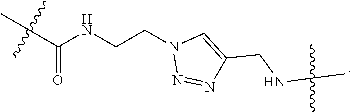 [SMILES: *NCc1cn(CCNC(*)=O)nn1]

Figures and examples illustrating the synthesis of compounds of Formula (VII) are shown in PCT International Application Publication No. WO 2020/139962, which is incorporated by reference herein in its entirety.

In some embodiments, the at least one E-selectin antagonist is a multimeric inhibitor of E-selectin, Galectin-3, and/or CXCR4, chosen from compounds of Formula (VIII):

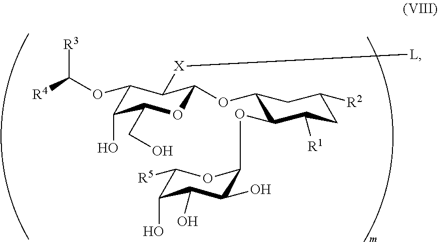

prodrugs of Formula (VIII), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, C₁₋₈ haloalkyl, C₂₋₈ haloalkenyl, C₂₋₈ haloalkynyl,

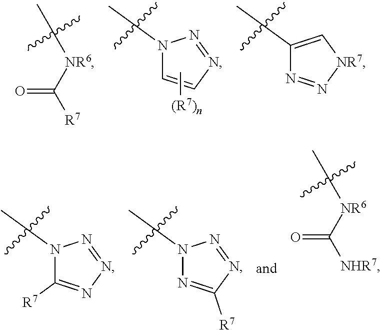

groups, wherein each n, which may be identical or different, is chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may be identical or different, is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

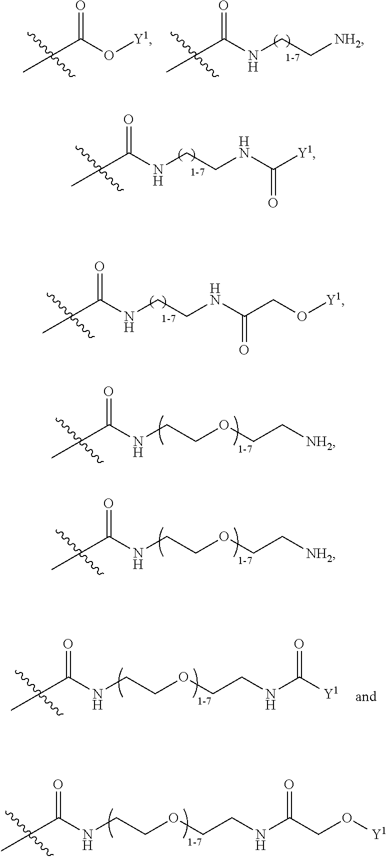

groups,
wherein each Y¹, which may be identical or different, is independently chosen from C₁₋₄ alkyl, C₂₋₄ alkenyl, and C₂₋₄ alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;
each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
m is chosen from integers ranging from 2 to 256; and
L is independently chosen from linker groups.

In some embodiments of Formula (VIII), at least one linker group is chosen from groups comprising spacer groups, such spacer groups as, for example, —(CH₂)_{z}— and —O(CH₂)_{z}—, wherein z is chosen from integers ranging from 1 to 250. Other non-limiting examples of spacer groups include carbonyl groups and carbonyl-containing groups such as, for example, amide groups. A non-limiting example of a spacer group is

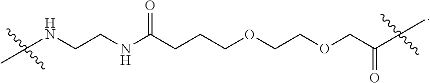 [SMILES: *NCCNC(=O)CCCOCCOCC(*)=O]

In some embodiments of Formula (VIII), at least one linker group is chosen from

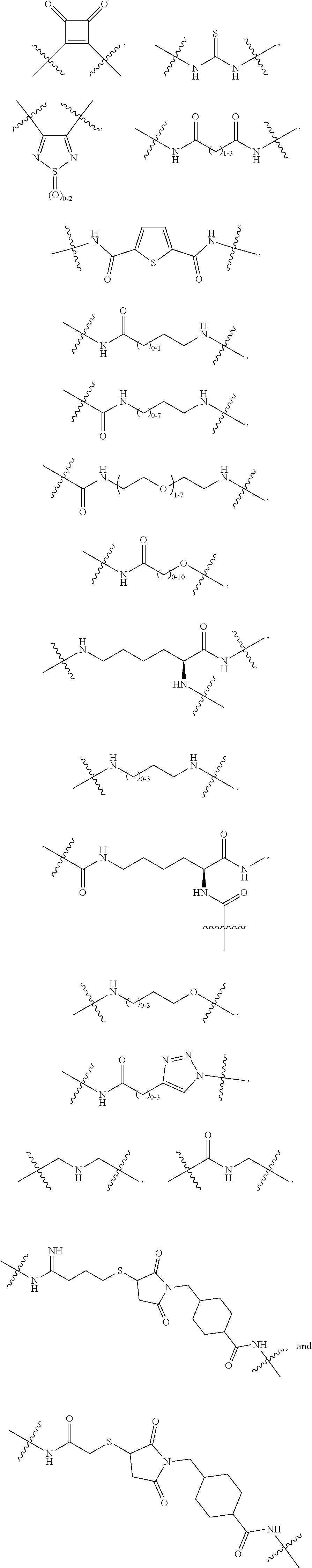

groups.

Other linker groups for certain embodiments of Formula (VIII), such as, for example, polyethylene glycols (PEGs) and —C(═O)—NH—(CH₂)_{z}—C(═O)—NH—, wherein z is chosen from integers ranging from 1 to 250, will be familiar to those of ordinary skill in the art and/or those in possession of the present disclosure.

In some embodiments of Formula (VIII), at least one linker group is

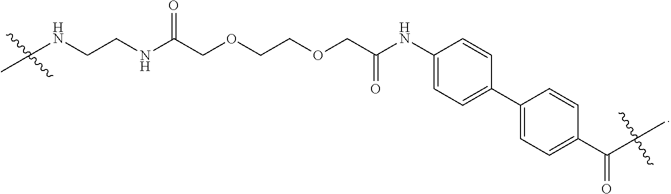 [SMILES: *NCCNC(=O)COCCOCC(=O)Nc1ccc(-c2ccc(C(*)=O)cc2)cc1]

In some embodiments of Formula (VIII), at least one linker group is

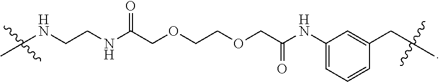 [SMILES: *Cc1cccc(NC(=O)COCCOCC(=O)NCCN*)c1]

In some embodiments of Formula (VIII), at least one linker group is chosen from —C(═O)NH(CH₂)₂NH—, —CH₂NHCH₂—, and —C(═O)NHCH₂—. In some embodiments of Formula (VIII), at least one linker group is —C(═O)NH(CH₂)₂NH—.

In some embodiments of Formula (VIII), L is chosen from dendrimers. In some embodiments of Formula (VIII), L is chosen from polyamidoamine (“PAMAM”) dendrimers. In some embodiments of Formula (VIII), L is chosen from PAMAM dendrimers comprising succinamic acid. In some embodiments of Formula (VIII), L is PAMAM GO generating a tetramer. In some embodiments of Formula (VIII), L is PAMAM G1 generating an octamer. In some embodiments of Formula (VIII), L is PAMAM G2 generating a 16-mer. In some embodiments of Formula (VIII), L is PAMAM G3 generating a 32-mer. In some embodiments of Formula (VIII), L is PAMAM G4 generating a 64-mer. In some embodiments, L is PAMAM G5 generating a 128-mer.

In some embodiments of Formula (VIII), m is 2 and L is chosen from

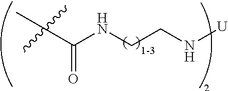

groups,
wherein U is chosen from

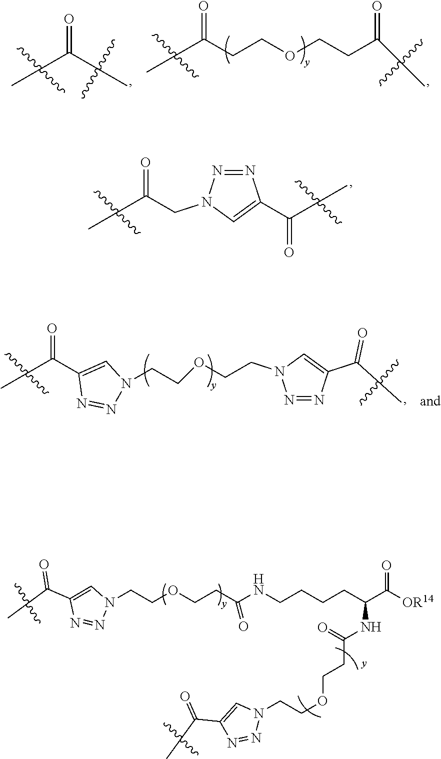

groups,
wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250. In some embodiments of Formula (VIII), R¹⁴ is chosen from C₁₋₈ alkyl. In some embodiments of Formula (VIII), R¹⁴ is chosen from C₇₋₁₉ arylalkyl. In some embodiments of Formula (VIII), R¹⁴ is H. In some embodiments of Formula (VIII), R¹⁴ is benzyl.

In some embodiments of Formula (VIII), L is chosen from

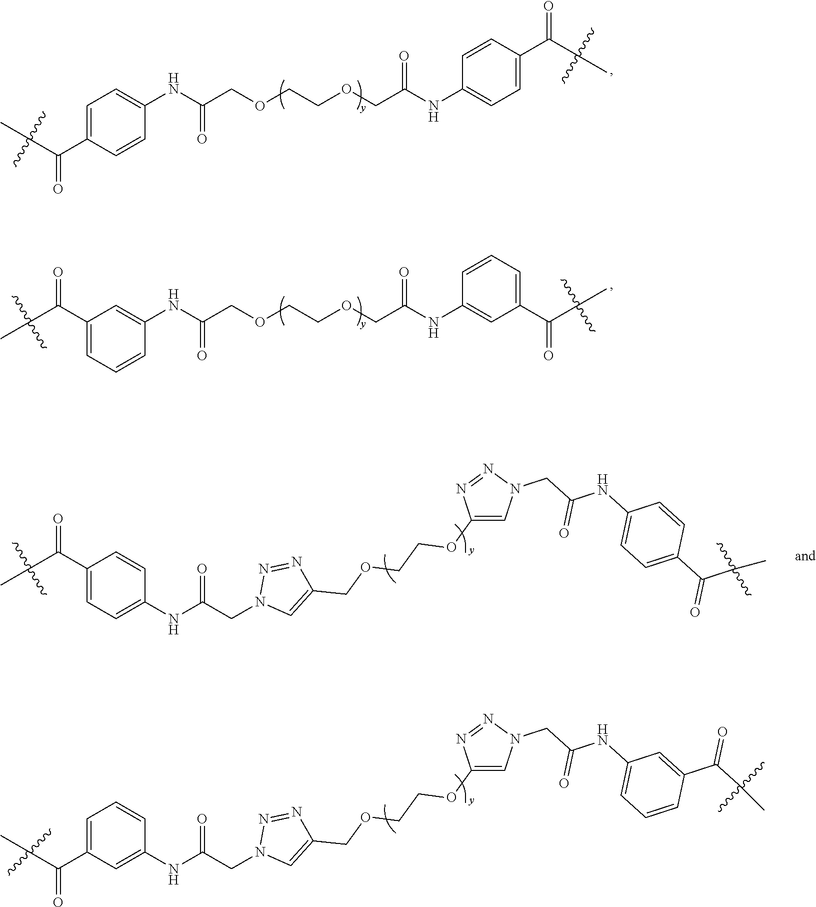

wherein y is chosen from integers ranging from 0 to 250.

In some embodiments of Formula (VIII), L is chosen from

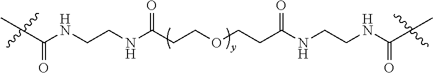

groups,
wherein y is chosen from integers ranging from 0 to 250.

In some embodiments of Formula (VIII), L is

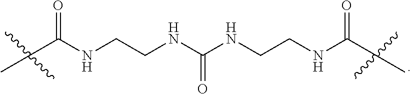 [SMILES: *C(=O)NCCNC(=O)NCCNC(*)=O]

In some embodiments of Formula (VIII), L is chosen from

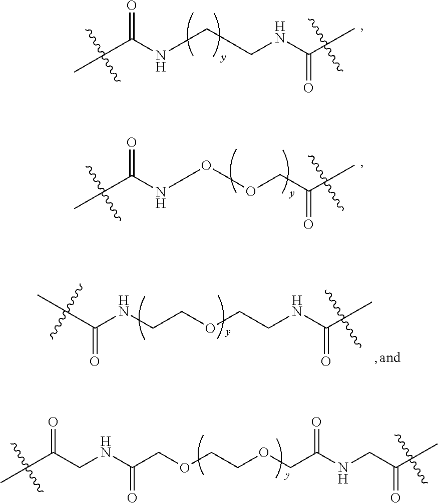

groups,
wherein y is chosen from integers ranging from 0 to 250.

In some embodiments of Formula (VIII), L is chosen from

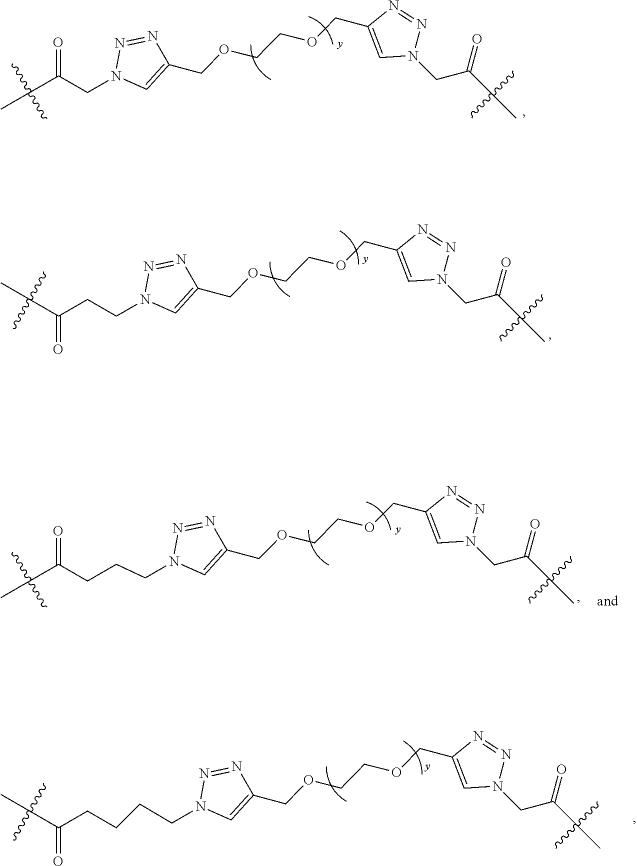

groups,
wherein y is chosen from integers ranging from 0 to 250.

In some embodiments of Formula (VIII), L is chosen from

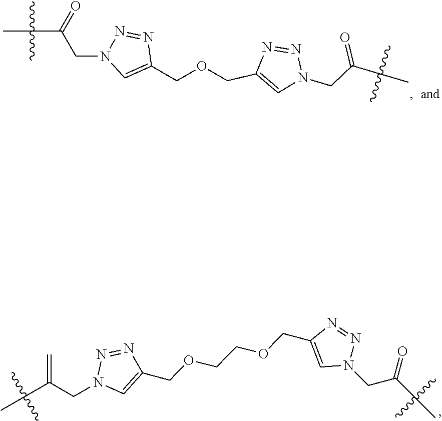 [SMILES: *C(=O)Cn1cc(COCc2cn(CC(*)=O)nn2)nn1 ; *C(=C)Cn1cc(COCCOCc2cn(CC(*)=O)nn2)nn1]

In some embodiments of Formula (VIII), L is

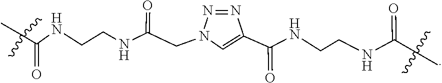 [SMILES: *C(=O)NCCNC(=O)Cn1cc(C(=O)NCCNC(*)=O)nn1]

In some embodiments of Formula (VIII), L is chosen from

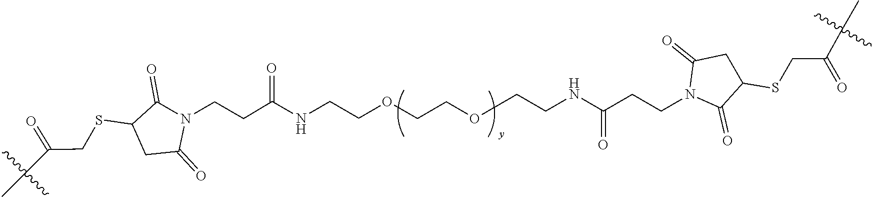

groups,
wherein y is chosen from integers ranging from 0 to 250.

In some embodiments of Formula (VIII), L is

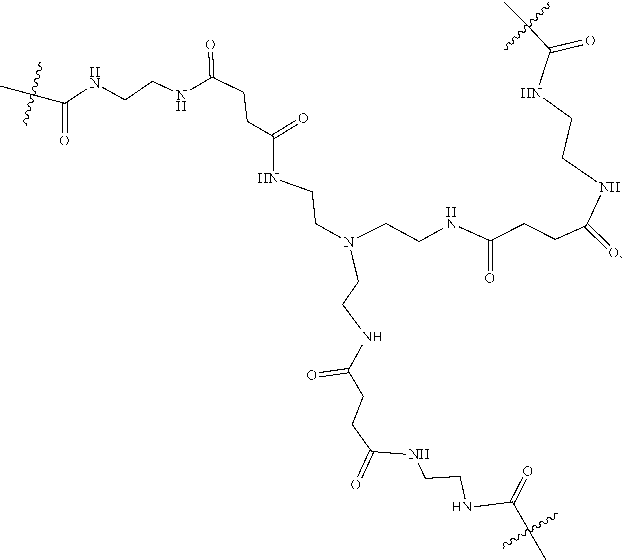 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments of Formula (VIII), L is

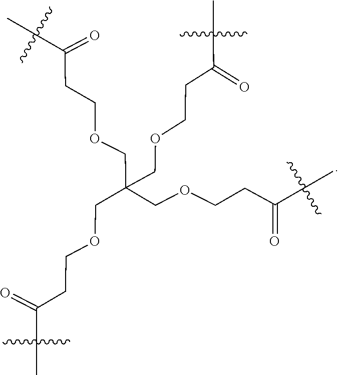 [SMILES: *C(=O)CCOCC(COCCC(*)=O)(COCCC(*)=O)COCCC(*)=O]

In some embodiments of Formula (VIII), L is

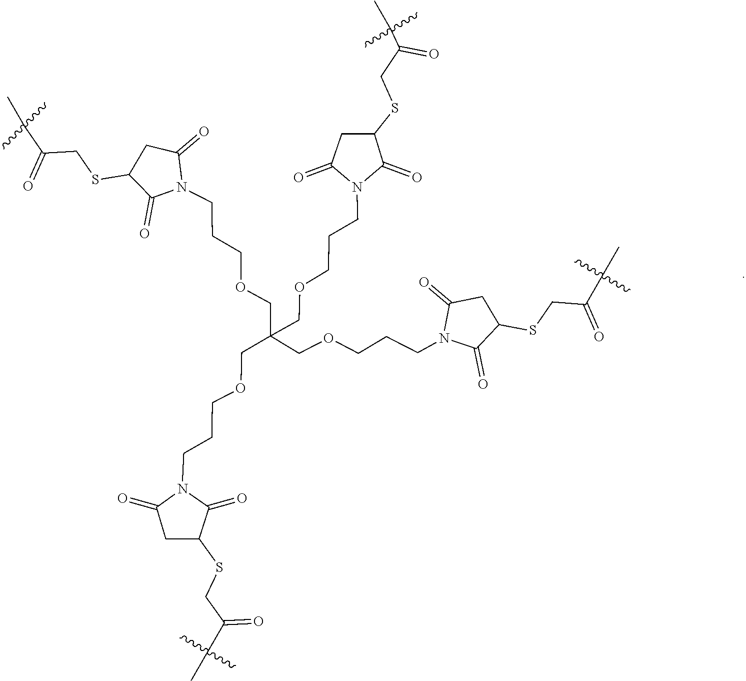 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments of Formula (VIII), L is chosen from

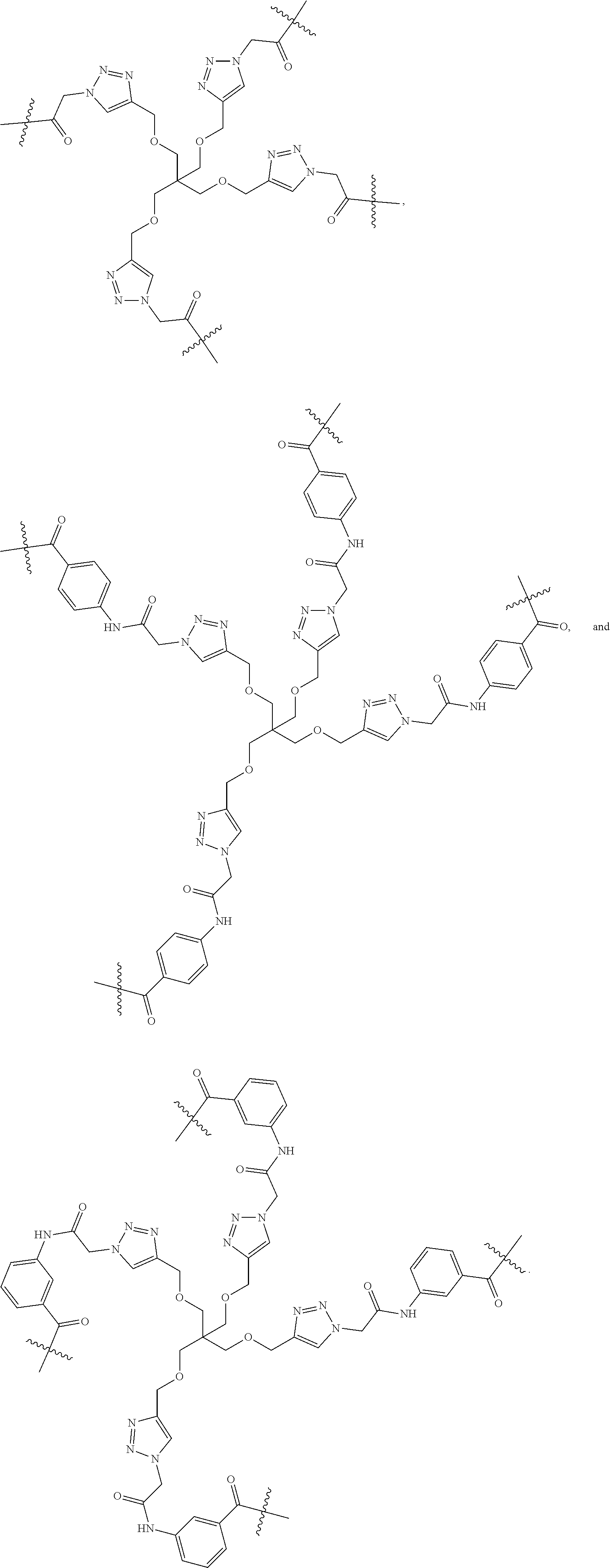 [SMILES: *C(=O)Cn1cc(COCC(COCc2cn(CC(*)=O)nn2)(COCc2cn(CC(*)=O)nn2)COCc2cn(CC(*)=O)nn2)nn1 ; *C(=O)c1ccc(NC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1 ; "and" ; *C(=O)c1cccc(NC(=O)Cn2cc(COCC(COCc3cn(CC(=O)Nc4cccc(C(*)=O)c4)nn3)(COCc3cn(CC(=O)Nc4cccc(C(*)=O)c4)nn3)COCc3cn(CC(=O)Nc4cccc(C(*)=O)c4)nn3)nn2)c1]

In some embodiments of Formula (VIII), L is

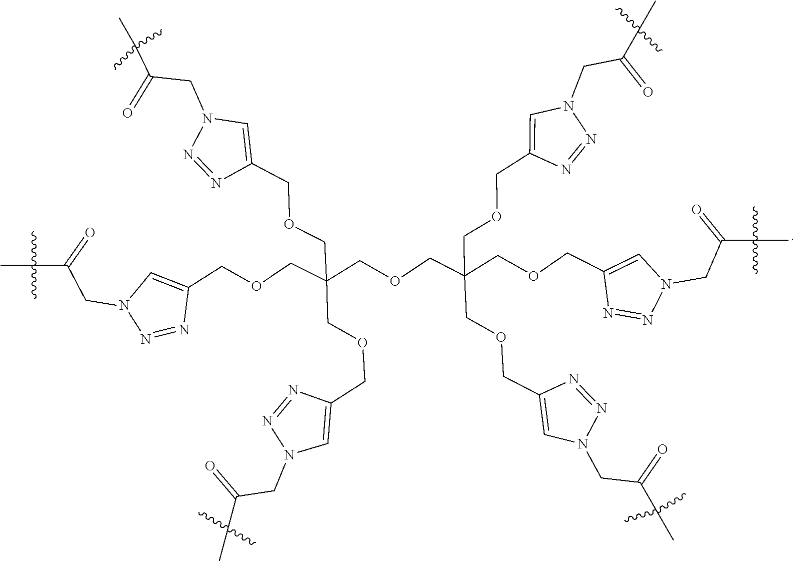 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments of Formula (VIII), L is chosen from

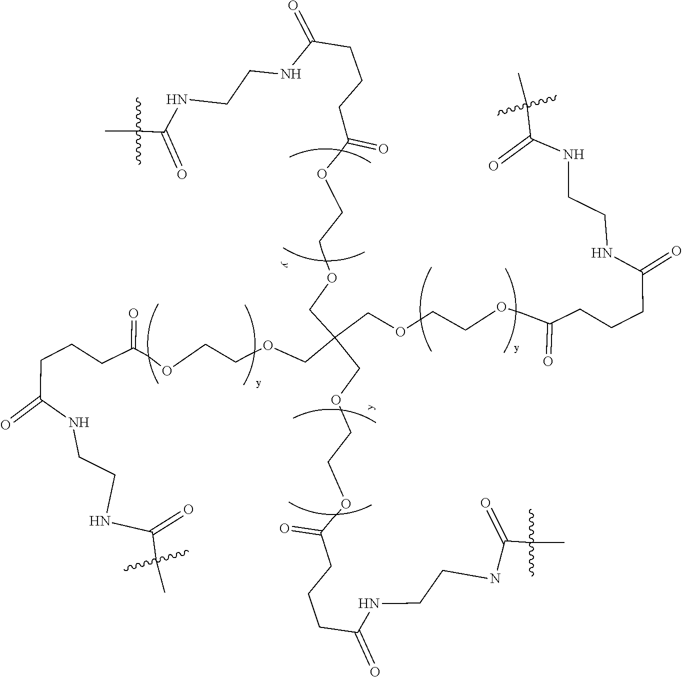

groups,
wherein each y, which may be identical or different, is independently chosen from integers ranging from 0 to 250.

In some embodiments of Formula (VIII), L is chosen from

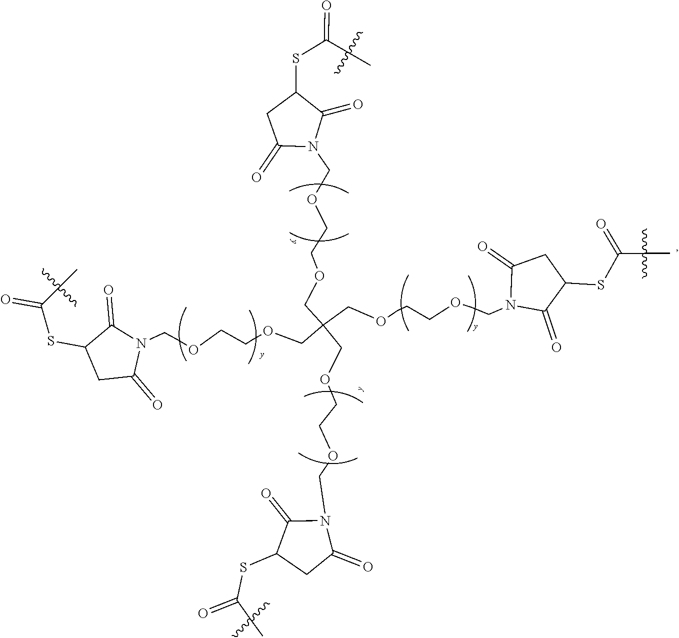

wherein each y, which may be identical or different, is independently chosen from integers ranging from 0 to 250.

In some embodiments of Formula (VIII), L is chosen from

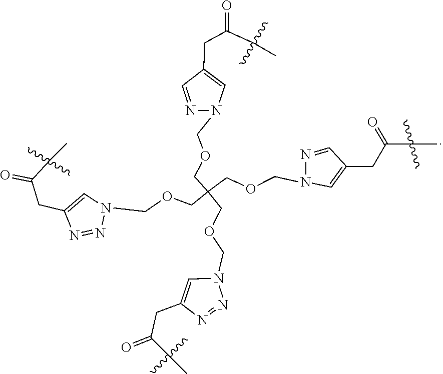

In some embodiments, at least one compound is chosen from compounds of Formula (VIII), wherein each R¹ is identical, each R² is identical, each R³ is identical, each R⁴ is identical, each R⁵ is identical, and each X is identical. In some embodiments, at least one compound is chosen from compounds of Formula (VIII), wherein said compound is symmetrical.

Figures and examples illustrating the synthesis of compounds of Formula (VIII) are shown in PCT International Application Publication No. WO 2020/219417, which is incorporated by reference herein.

Also provided are pharmaceutical compositions comprising at least one E-selectin antagonist chosen from compounds of Formula (I), (Ia), (II), (IIa), (III), (IIIa), (IV), (V), (IVa/Va), (IVb/Vb), (VI), (VII), and (VIII). These compounds and compositions may be used in the methods described herein. In some embodiments, provided are pharmaceutical compositions comprising at least one E-selectin antagonist chosen from Compound A, Compound B, Compound C, Compound D, and Compound E. These compounds and compositions may be used in the methods described herein.

Also provided are pharmaceutical compositions comprising at least one pharmaceutically acceptable excipient and at least one E-selectin antagonist chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing. In some embodiments, provided are pharmaceutical compositions comprising at least one pharmaceutically acceptable excipient and at least one E-selectin antagonist chosen from Compound A, Compound B, Compound C, Compound D, and Compound E, and pharmaceutically acceptable salts of any of the foregoing. These compounds and compositions may be used in the methods described herein.

In some embodiments, the at least one E-selectin antagonist is chosen from compounds of Formula (I), (Ia), (II), (IIa), (III), (IIIa), (IV), (V), (IVa/Va), (IVb/Vb), (VI), (VII), and (VIII) and pharmaceutically acceptable salts of any of the foregoing. In some embodiments, the at least one E-selectin antagonist is chosen from compounds of Formula (I), (Ia), (II), (IIa), (III), (IIIa), (IV), (V), (IVa/Va), (IVb/Vb), (VI), (VII), and (VIII). In some embodiments, the at least one E-selectin antagonist is Compound A. In some embodiments, the at least one E-selectin antagonist is Compound B. In some embodiments, the at least one E-selectin antagonist is Compound C. In some embodiments, the at least one E-selectin antagonist is Compound D. In some embodiments, the at least one E-selectin antagonist is Compound E.

In some embodiments, the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of the at least one E-selectin antagonist. In some embodiments, the method comprises administering a dose in the range of 5 mg/kg to 100 mg/kg (such as, e.g., 5 mg/kg, 10 mg/kg, 15 mg/kg, 20 mg/kg, 25 mg/kg, 30 mg/kg, 35 mg/kg, 40 mg/kg, 45 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg; e.g., 5 mg/kg to 50 mg/kg, 10 mg/kg to 30 mg/kg, 10 mg/kg to 50 mg/kg, etc.) of Compound A.

In some embodiments, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of the at least one E-selectin antagonist.

In some embodiments, the method comprises administering a fixed dose of 20 mg to 4000 mg (such as, e.g., 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, e.g., 800 mg to 3200 mg per day, 1000 mg to 2000 mg per day) per day of Compound A.

In some embodiments, the at least one antineoplastic agent is chosen from chemotherapeutic agents. In some embodiments, the at least one antineoplastic agent is chosen from mitoxantrone, etoposide, and cytarabine. In some embodiments, the at least one antineoplastic agent is mitoxantrone, etoposide, and cytarabine. In some embodiments, the at least one antineoplastic agent is mitoxantrone. In some embodiments, the at least one antineoplastic agent is etoposide. In some embodiments, the at least one antineoplastic agent is cytarabine. In some embodiments, the at least one antineoplastic agent is daunomycin. In some embodiments, the at least one antineoplastic agent is idarubicin.

In some embodiments, the at least one antineoplastic agent is chosen from targeted therapy drugs. In some embodiments, the at least one antineoplastic agent is chosen from tretinoin, imatinib mesylate, dasatinib, nilotinib, bosutinib, rituximab, alemtuzumab, ofatumumab, obinutuzumab, ibrutinib, idelalisib, blinatumomab, venetoclax, ponatinib hydrochloride, midostaurin, enasidenib mesylate, inotuzumab ozogamicin, tisagenlecleucel, gemtuzumab ozogamicin, rituximab and hyaluronidase human, ivosidenib, duvelisib, moxetumomab pasudotox-tdfk, glasdegib maleate, gilteritinib, tagraxofusp-erzs, and acalabrutinib.

In some embodiments, the at least one antineoplastic agent is venetoclax.

In some embodiments, the method comprises administering a fixed dose of 10 mg to 1000 mg (such as, e.g., 10 mg, 20 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1000 mg, e.g., 20 mg to 400 mg) per day of venetoclax. In some embodiments, the method comprises administering a fixed dose of 400 mg per day of venetoclax.

In some embodiments, the at least one hypomethylating agent is chosen from 5-azacitidine, 5-aza-2′-deoxycytidine (decitabine), guadecitabine, 5-fluoro-2′-deoxycytidine, zebularine, CP-4200, RG108, and nanaomycin A. In some embodiments, the at least one hypomethylating agent is chosen from 5-azacitidine, decitabine, guadecitabine, 5-fluoro-2′-deoxycytidine, and zebularine. In some embodiments, the at least one hypomethylating agent is chosen from 5-azacitidine and decitabine.

In some embodiments, the at least one hypomethylating agent is 5-azacitidine.

In some embodiments, the at least one hypomethylating agent is decitabine.

The E-selectin ligand glycosylation genes, FUT7 and ST3GAL4 are consistently expressed in the majority of cancer subtypes. The top five cancer types, based in mean expression:
FUT7: Acute Myeloid Leukemia (LAML), Lymphoid Neoplasm Diffuse Large B cell Lymphoma (DBLC), Thymoma (THYM), Testicular Germ Cell Tumors (TGCT), and Head and Neck Squamous Cell Carcinoma (HNSC);
ST3GAL4: Uveal Melanoma (UVM), Skin Cutaneous Melanoma (SKCM), Kidney Chromophobe (KICH), Adrenocortical Carcinoma (ACC), and Bladder Urothelial Carcinoma.

The E-selectin ligand glycosylation genes, FUT7 and ST3GAL4, are also consistently expressed in tumor cell lines comprising the Cancer Cell Line Encyclopedia database. The top five cancer types, based on mean expression:
FUT7: T-cell Lymphoma, AML, B-cell Acute Lymphoblastic Leukemia, Other Leukemias and Chronic Myelogenous Leukemia (CML);
ST3GAL4: Melanoma, AML, CML, Pancreas, and Breast.

In some embodiments, the cancer is chosen from liquid cancers.

In some embodiments, the cancer is chosen from solid cancers.

In some embodiments, the cancer is chosen from AML, lymphoid neoplasm diffuse large B cell lymphoma, thymoma, testicular germ cell tumors, and head and neck squamous cell carcinoma.

In some embodiments, the cancer is chosen from T-cell lymphoma, AML, B-cell acute lymphoblastic leukemia, chronic myelogenous leukemia.

In some embodiments, the cancer is chosen from uveal melanoma, skin cutaneous melanoma, kidney chromophobe, adrenocortical carcinoma, and bladder urothelial carcinoma.

In some embodiments, the cancer is chosen from melanoma, AML, CML, pancreatic cancer, and breast cancer.

In some embodiments, the cancer is chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

In some embodiments, the cancer is chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

In some embodiments, the leukemia is chosen from acute myeloid leukemia, acute lymphocytic leukemia, chronic lymphocytic leukemia, and chronic myelogenous leukemia.

In some embodiments, the lymphoma is chosen from non-Hodgkin's lymphoma and Hodgkin's lymphoma.

In some embodiments, the myeloma is multiple myeloma.

In some embodiments, the melanoma is chosen from uveal melanoma and skin melanoma.

In some embodiments, the cancer is chosen from FLT3 mutated cancers. In some embodiments, the cancer is chosen from FLT3-ITD mutated cancers.

In some embodiments, the cancer is AML. In some embodiments, the cancer is relapsed/refractory AML. In some embodiments, the cancer is FLT3-ITD mutated AML.

In some embodiments, the subject has acquired resistance to a therapy comprising at least one antineoplastic agent. In some embodiments, the subject has acquired resistance to a therapy comprising venetoclax. In some embodiments, the subject has acquired resistance to a therapy comprising sorafenib.

In some embodiments, the subject has acquired resistance to a therapy comprising at least one hypomethylating agent. In some embodiments, the subject has acquired resistance to a therapy comprising 5-azacitidine. In some embodiments, the subject has acquired resistance to a therapy comprising decitabine.

In some embodiments, the subject has acquired resistance to a combination therapy comprising at least one antineoplastic agent and at least one hypomethylating agent. In some embodiments, the subject has acquired resistance to a combination therapy comprising venetoclax and at least one hypomethylating agent. In some embodiments, the subject has acquired resistance to a combination therapy comprising venetoclax and 5-azacitidine. In some embodiments, the subject has acquired resistance to a combination therapy comprising venetoclax and decitabine.

In some embodiments, the subject possesses one or more mutational alterations of FLT3. In some embodiments, the mutational alterations are chosen from internal tandem duplications and missense mutations within the tyrosine kinase domain activation loop of FLT3. In some embodiments, the mutational alterations are chosen from internal tandem duplications within the tyrosine kinase domain activation loop of FLT3. In some embodiments, the mutational alterations are chosen from missense mutations within the tyrosine kinase domain activation loop of FLT3.

In some embodiments, the subject expresses the gene ST3GAL4 at an expression level greater than that of 55%, 60%, 65%, 70%, 75%, 80%, 85%, 90%, or 95% of cancer patients. In some embodiments, the subject expresses the gene B3GNT5 at an expression level greater than that of 55%, 60%, 65%, 70%, 75%, 80%, 85%, 90%, or 95% of cancer patients. In some embodiments, the subject expresses the gene FUT5 at an expression level greater than that of 55%, 60%, 65%, 70%, 75%, 80%, 85%, 90%, or 95% of cancer patients. In some embodiments, the subject expresses the gene FUT7 at an expression level greater than that of 55%, 60%, 65%, 70%, 75%, 80%, 85%, 90%, or 95% of cancer patients. In some embodiments, the subject expresses the genes ST3GAL4 and FUT5 at an expression level greater than that of 55%, 60%, 65%, 70%, 75%, 80%, 85%, 90%, or 95% of cancer patients. In some embodiments, the subject expresses the genes ST3GAL4 and FUT7 at an expression level greater than that of 55%, 60%, 65%, 70%, 75%, 80%, 85%, 90%, or 95% of cancer patients. In some embodiments, the subject expresses the genes FUT5 and FUT7 at an expression level greater than that of 55%, 60%, 65%, 70%, 75%, 80%, 85%, 90%, or 95% of cancer patients. In some embodiments, the subject expresses the genes ST3GAL4, FUT5, and FUT7 at an expression level greater than that of 55%, 60%, 65%, 70%, 75%, 80%, 85%, 90%, or 95% of cancer patients.

In some embodiments, the subject expresses the gene ST3GAL4 at an expression level greater than that of 55%, 60%, 65%, 70%, 75%, 80%, 85%, 90%, or 95% of patients with relapsed/refractory AML. In some embodiments, the subject expresses the gene B3GNT5 at an expression level greater than that of 55%, 60%, 65%, 70%, 75%, 80%, 85%, 90%, or 95% of patients with relapsed/refractory AML. In some embodiments, the subject expresses the gene FUT5 at an expression level greater than that of 55%, 60%, 65%, 70%, 75%, 80%, 85%, 90%, or 95% of patients with relapsed/refractory AML. In some embodiments, the subject expresses the gene FUT7 at an expression level greater than that of 55%, 60%, 65%, 70%, 75%, 80%, 85%, 90%, or 95% of patients with relapsed/refractory AML. In some embodiments, the subject expresses the genes ST3GAL4 and FUT5 at an expression level greater than that of 55%, 60%, 65%, 70%, 75%, 80%, 85%, 90%, or 95% of patients with relapsed/refractory AML. In some embodiments, the subject expresses the genes ST3GAL4 and FUT7 at an expression level greater than that of 55%, 60%, 65%, 70%, 75%, 80%, 85%, 90%, or 95% of patients with relapsed/refractory AML. In some embodiments, the subject expresses the genes FUT5 and FUT7 at an expression level greater than that of 55%, 60%, 65%, 70%, 75%, 80%, 85%, 90%, or 95% of patients with relapsed/refractory AML. In some embodiments, the subject expresses the genes ST3GAL4, FUT5, and FUT7 at an expression level greater than that of 55%, 60%, 65%, 70%, 75%, 80%, 85%, 90%, or 95% of patients with relapsed/refractory AML.

Gene expression may also be measured by the amount of protein in a patient sample. Non-limiting example methods to measure the amount of protein include but are not limited to immunostaining, immunohistochemistry, affinity purification, mass spectrometry, Western blotting, and enzyme-linked immunosorbent assay (ELISA).

In some embodiments, gene expression level is measured by the amount of mRNA.

In some embodiments, gene expression level is measured by the amount of protein in a patient sample.

In some embodiments, the method further comprises selecting the subject to treat through a method comprising: (a) determining or having determined the gene expression level of one or more genes in the subject or a sample from the subject; and (b) selecting the subject for treatment when at least 10% of the blast cells from the subject or sample from the subject expresses the one or more genes. In some embodiments, the one or more genes are chosen from ST3GAL4, B3GNT5, and FUT7. In some embodiments, gene expression level is measured by the amount of mRNA. In some embodiments, gene expression level is determined by high coverage single-strand mRNA sequencing. In some embodiments, gene expression level is measured by the amount of protein in the sample from the subject. In some embodiments, the sample from the subject is peripheral blood.

In some embodiments, the method further comprises selecting the subject to treat through a method comprising: (a) obtaining or having obtained a biological sample comprising blast cells from the subject; (b) performing or having performed an assay on the biological sample to determine the gene expression level of one or more E-selectin ligand-forming genes in the sample; and (c) selecting the subject for treatment when at least 10% of the blast cells in the sample express the one or more E-selectin ligand-forming genes.

In some embodiments, the biological sample is a bone marrow sample. In some embodiments, the biological sample is a peripheral blood sample.

In some embodiments, the one or more E-selectin ligand-forming genes are glycosylation genes. In some embodiments, the one or more E-selectin-ligand forming genes are chosen from ST3GAL3, ST3GAL4, FUCA2, FUT5, and FUT7. In some embodiments, the one or more E-selectin-ligand forming genes are chosen from ST3GAL4, FUT5, and FUT7. In some embodiments, the one or more E-selectin-ligand forming genes are chosen from ST3GAL4 and FUT7. In some embodiments, at least one of the one or more E-selectin-ligand forming genes is ST3GAL4. In some embodiments, at least one of the one or more E-selectin-ligand forming genes is FUT7.

In some embodiments, the method further comprises selecting the subject to treat through a method comprising: (a) determining the gene expression level of one or more genes in the subject or a sample from the subject; (b) comparing the gene expression level from (a) to a control sample from a cancer-free subject, a newly diagnosed cancer subject, or a subject diagnosed with the same cancer as the subject, and (c) selecting the subject for treatment when the gene expression level exceeds that in the control sample. In some embodiments, the one or more genes are chosen from ST3GAL4, B3GNT5, and FUT7. In some embodiments, gene expression level is measured by the amount of mRNA. In some embodiments, gene expression level is determined by high coverage single-strand mRNA sequencing. In some embodiments, gene expression level is measured by the amount of protein in the sample from the subject. In some embodiments, the sample from the subject is peripheral blood.

In some embodiments, the method further comprises determining the presence of one or more mutational alterations of FLT3. In some embodiments, the mutational alterations are chosen from internal tandem duplications and missense mutations within the tyrosine kinase domain activation loop of FLT3.

### EXAMPLES

The following examples are intended to be illustrative and are not meant in any way to limit the scope of the disclosure.

### Example 1

To determine if E-selectin has indispensable effects in bone marrow niche component cells, healthy donor derived-mesenchymal stroma cells (MSC) were exposed to increasing concentrations of E-selectin. Soluble E-selectin upregulated the surface expression of the most potent E-selectin ligand, CD44, in human MSC. Abrogation of E-selectin binding by Compound A diminished CD44 expression in vitro.

Targeting E-selectin with Compound A (50 mg/kg) attenuated phosphorylation of the enzyme eNOS in HUVECs co-cultured with AML cells, suggesting that E-selectin inhibition may protect disruption of BM vasculatures during AML progression.

### Example 2

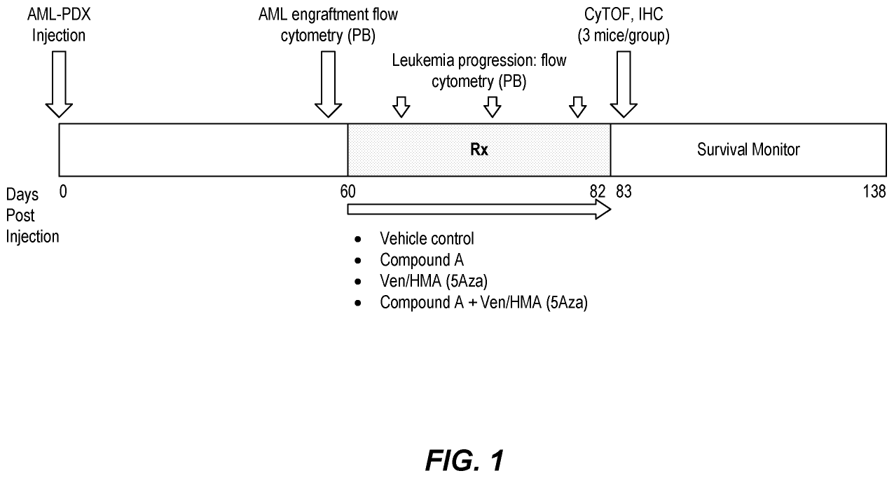

To evaluate the efficacy of targeting E-selectin with Compound A to selectively eradicate leukemia cells resistant to venetoclax/HMA therapy in the bone marrow niche, an in vivo PDX-AML model derived from an AML patient harboring FLT3-ITD, NRAS, and GATA2 mutations who initially responded to venetoclax/HMA therapy and then relapsed was employed (FIG. 1). The model reflects the present situation for many elderly AML patients: initial sensitivity, followed by resistance to venetoclax/HMA and relapse.

Patient-derived PDX cells from an AML patient (2.5×10⁶ cells/mouse) were transplanted via tail vein into NSG mice. Once AML cells began to engraft, mice were divided into four groups: vehicle treatment only; 40 mg/kg of Compound A; 50 mg/kg venetoclax+5.5 mg/kg 5-azacitidine; and a combination of 40 mg/kg Compound A and 50 mg/kg venetoclax+5.5 mg/kg 5-azacitidine. Drug treatment was performed from day 60 to day 82 post-transplantation.

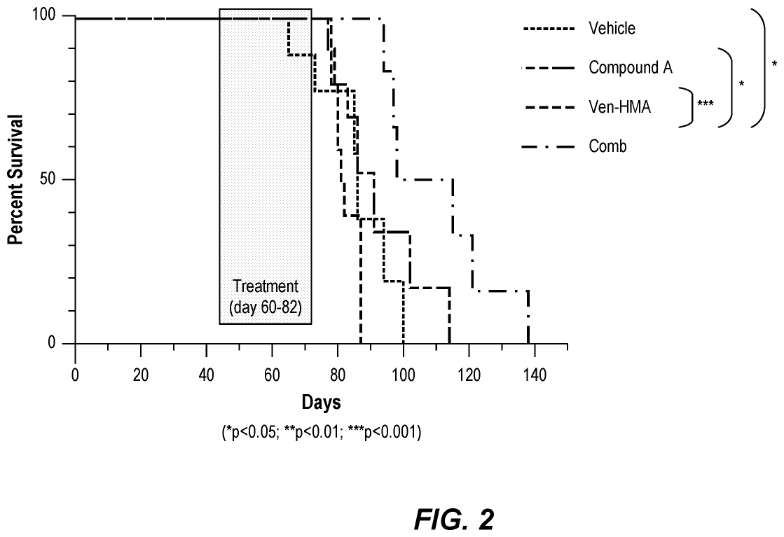

Leukemia progression and tumor burden were evaluated weekly during the treatment period (for 22 days) by determining the frequency and absolute number of human CD45⁺ cells in peripheral blood using flow cytometry analysis. The synergistic effects of the combinatorial treatment on AML-PDX mouse survival were determined by Kaplan-Meier analysis (FIG. 2). The combination of Compound A and venetoclax/HMA statistically significantly prolonged the survival of mice compared to vehicle control (p=0.015) as well as the venetoclax/HMA (p=0.0009) and Compound A groups (p=0.03). The median survival of the vehicle control, Compound A, venetoclax/HMA, and combination-treated (Compound A+venetoclax/HMA) groups of mice was 86, 91, 81.5, and 106.5 days, respectively.

At the time when all of the control group of mice were moribund (after 23 days of treatment), 3 mice per group were sacrificed for single cell proteomics (CyTOF) and immunohistochemistry analysis.

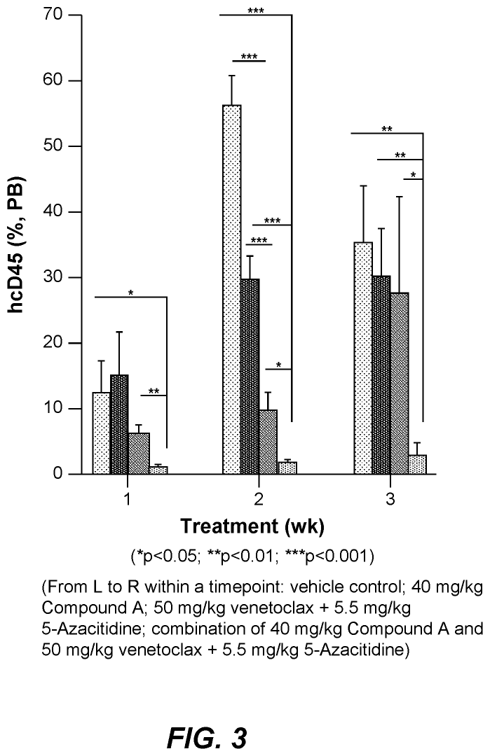

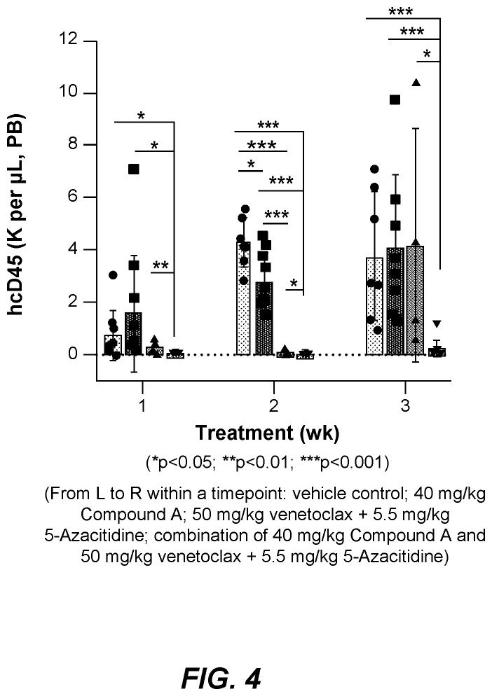

Targeting E-selectin with Compound A mobilized human AML cells and sensitized them to venetoclax/HMA. The number of circulating leukemic cells was significantly reduced by combinatorial treatment of Compound A with venetoclax/HMA compared to venetoclax/HMA alone (p<0.05) (FIGS. 3, 4).

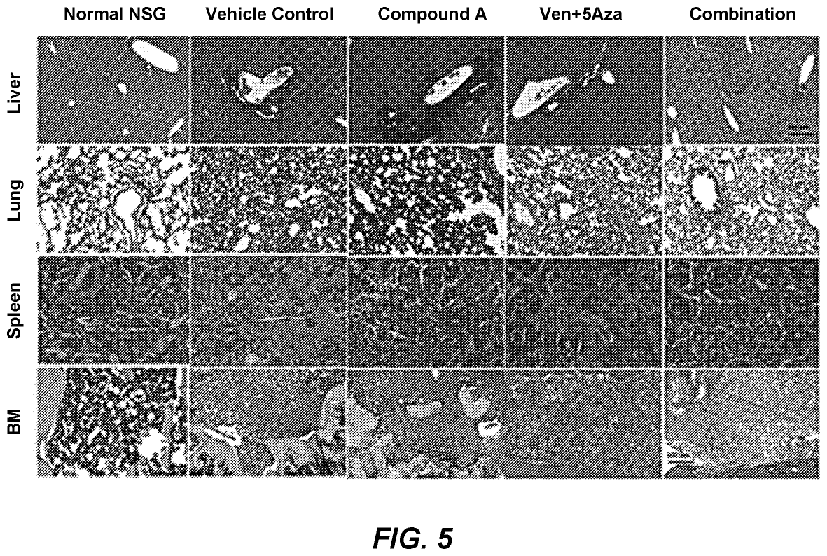

Histological analysis of bone marrow, spleen, lung, and liver demonstrated differences in leukemia cell infiltration, confirming enhanced anti-leukemia efficacy of the combination treatment (FIG. 5). Compared to normal NSC control mice, leukemia cell infiltrations were increased in the organs of mice treated with vehicle control or Compound A only. However, mice treated with a combination of Compound A and venetoclax/HMA exhibited a reduction in leukemia cell infiltration, indicating that inhibition of E-selectin improves the therapeutic efficacy of venetoclax/HMA in this drug-resistant AML-PDX model.

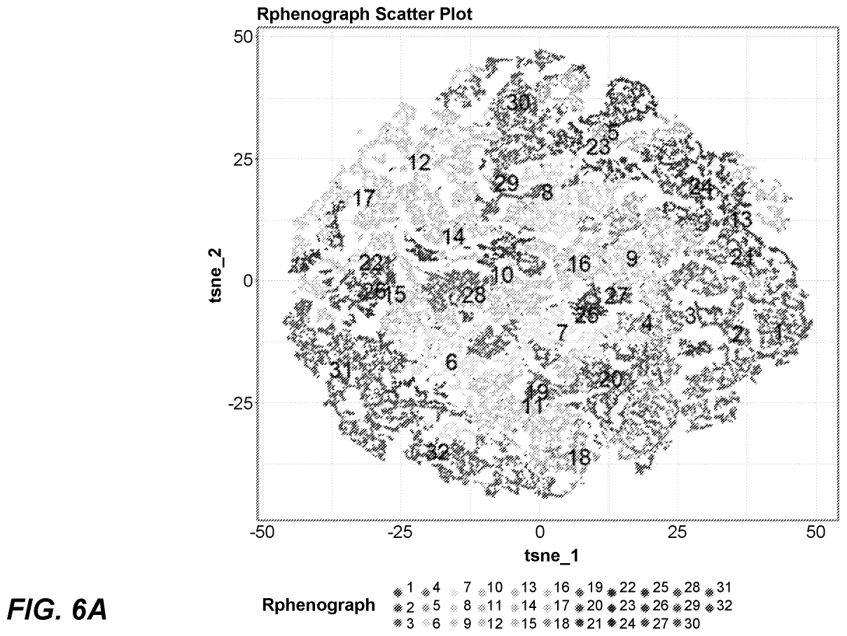

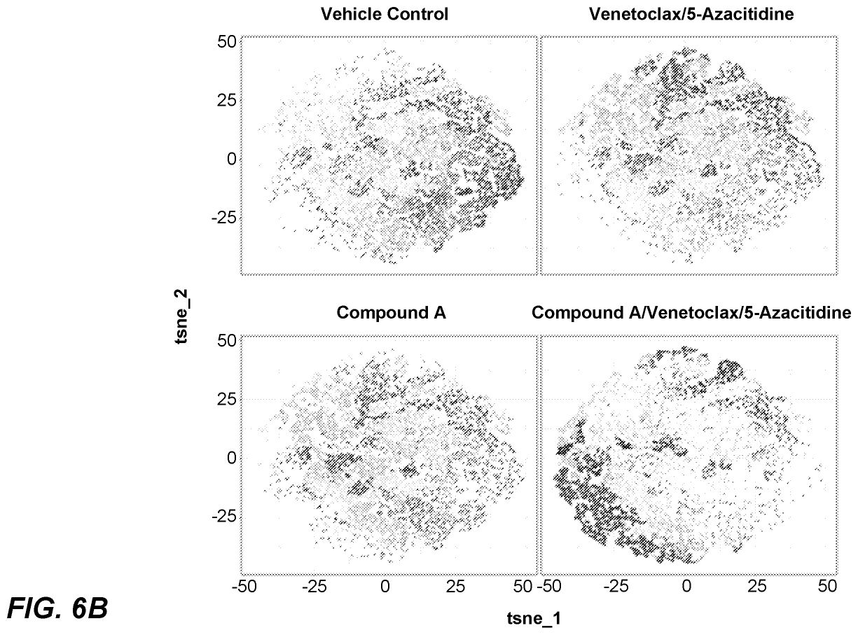

To identify intrinsic and extrinsic molecular mechanisms associated with enhanced efficacy induced by E-selectin inhibition, single cell proteomics using CyTOF was performed. FIG. 6A displays all the clusters of human CD45⁺ cells.

The LSC population was identified by four surface markers (CD34, CD123, CD45, and CD38). CD45⁺ CD34⁺ CD38⁻ CD123⁺LSC populations were represented by clusters 20 and 25. Co-targeting E-selectin and Bcl-2 with HMA treatment efficiently eliminated clusters 20 and 25 LSC populations (FIG. 6B).

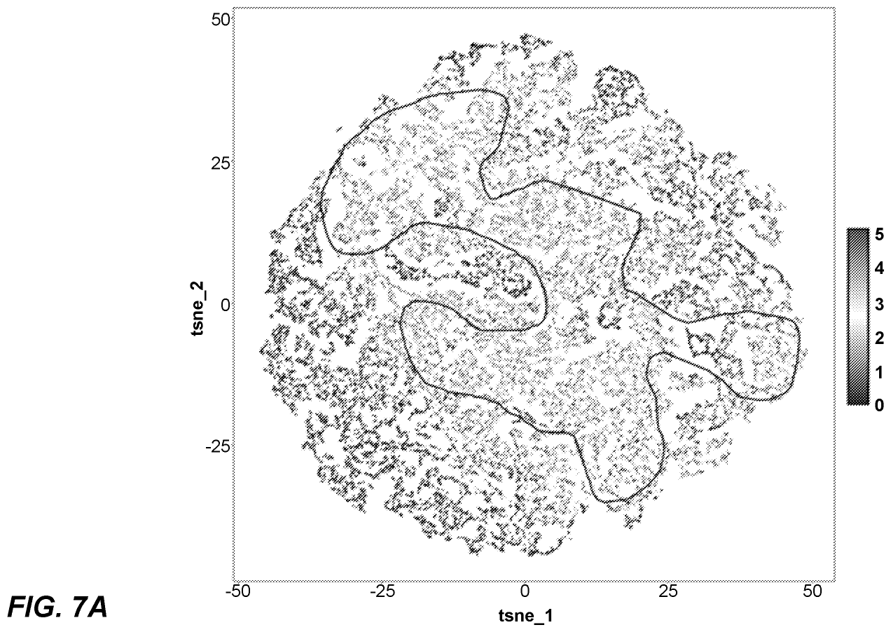

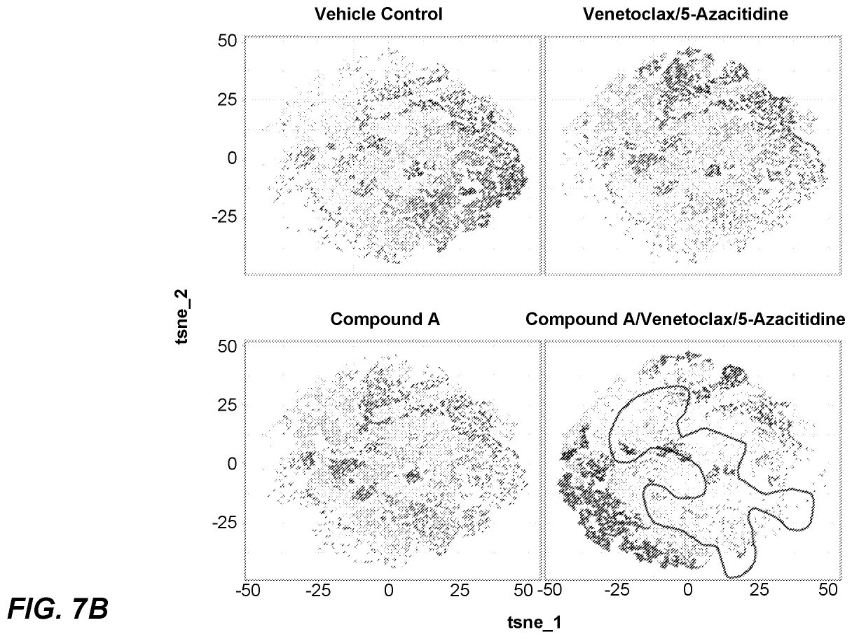

High E-selectin-binding potential (as represented by high E-selectin ligand expression) distinguishes chemo-resistant AML blasts. In this study, most venetoclax/HMA resistant cells expressed higher level of E-selectin ligand, including LSC clusters. In vivo administration of Compound A enhanced the anti-leukemia efficacy of venetoclax/HMA, as demonstrated by high E-selectin ligand expression in the overall cluster TSNE map (FIG. 7A) and the elimination of AML cells in the combination treatment group (FIG. 7B).

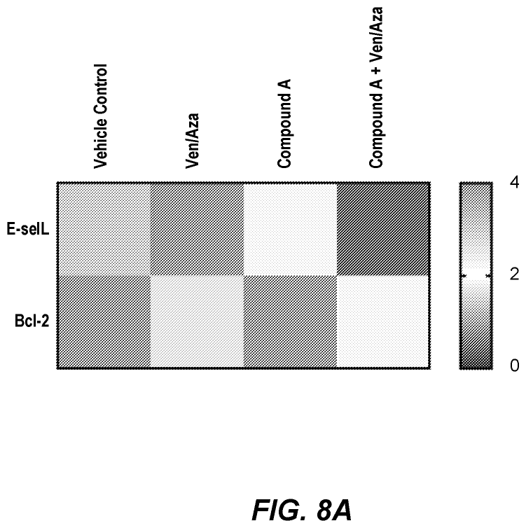

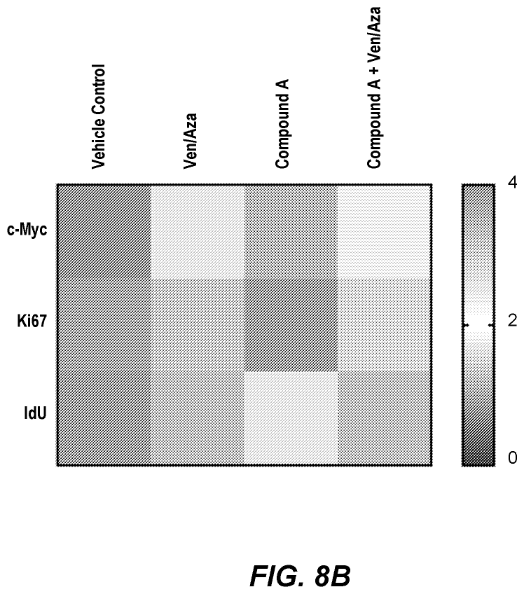

The degree of AML proliferation was also assessed across treatment groups. Levels of c-Myc, Ki67, and IdU positivity all decreased in combination therapy treated mice, suggesting that inhibition of E-selectin further decreases proliferation in residual cells after venetoclax/HMA treatment (FIG. 8B).

### Example 3

To delineate the mechanism of E-selectin at the onset of drug-mediated changes in AML signaling signatures, another PDX model was employed (Flt3-ITD and WT1 mutations, sorafenib-resistant).

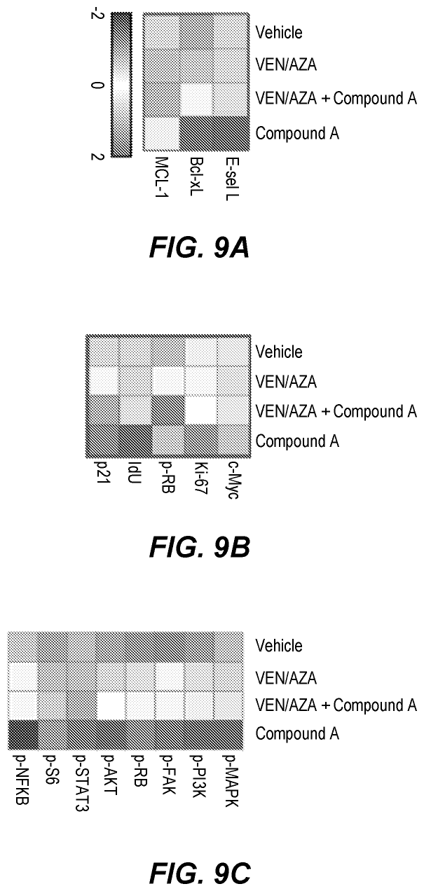

PDX mice with advanced AML (more than 20% human AML cells circulation in peripheral blood) were administered vehicle control, venetoclax (25 mg/kg)/HMA (5.5 mg/kg), Compound A (200 mg/kg), or a combination therapy for 2 days. After 2 days of bolus drug administration, mice were sacrificed and subjected to CyTOF analysis (FIGS. 9A-C). Single cell proteomics analysis by CyTOF determined that combinatorial treatment of Compound A with venetoclax/HMA diminished levels of Ki67, IDU, and pRb compared to vehicle control or venetoclax/HMA alone, resulting in decreased proliferation of AML blasts.

It has recently been reported that venetoclax-resistant AML cells exhibit an increased dependence on alternate anti-apoptotic proteins, Mcl-1 and Bcl-xl (Konopleva et al., 2016). In this example, concomitant treatment in vivo with Compound A and venetoclax/HMA further decreased the expression of Bcl-xl and Mcl-1 in AML blasts compared to Ven/HMA alone, suggesting a critical role for E-selectin antagonists in overcoming drug resistance.

E-selectin binding potential and focal adhesion kinase activity in AML blasts were decreased upon acute administration of pharmacological E-selectin inhibitor. Other oncogenic signaling pathways interrogated, including MAPK, p-S6, and STAT3, were all inhibited by the addition of Compound A to venetoclax/HMA.

Activation of eNOS to produce nitric oxide (NO) through PI3K/AKT kinase maintains clonogenic cell growth in malignant cells. A recent publication has demonstrated that introduction of NOS blockers in combination with chemotherapy led to slower leukemia progression and longer remissions in contrast to chemotherapy alone (Passaro et al, 2017).

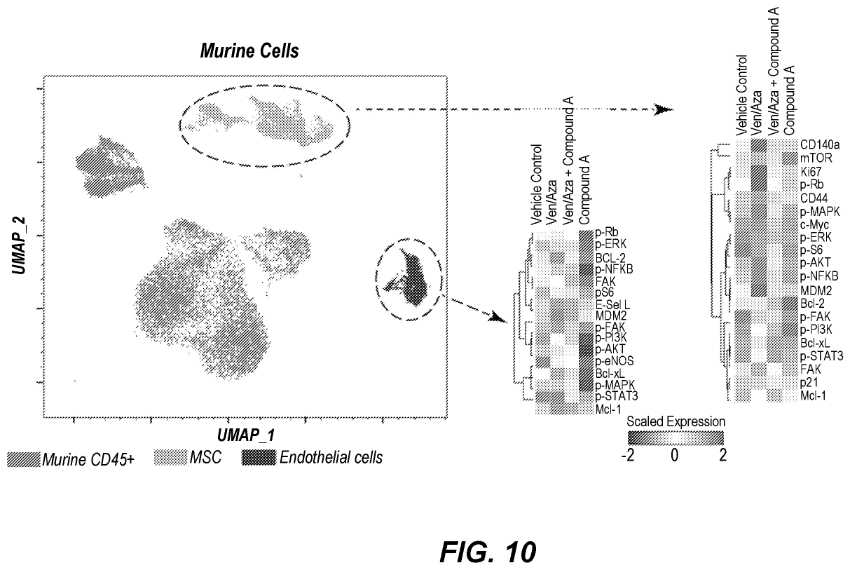

In this study, reduced activation of PI3K and AKT was observed in AML blasts as well as in BM CD31⁺EC cells in the Compound A-treated PDX model (FIG. 10). eNOS phosphorylation was subsequently decreased in EC, suggesting that inhibition of E-selectin may protect BM vasculature by blocking the production of NO. In addition, targeting E-selectin showed signaling alterations in AML-derived MSC (FIG. 10). Administration of E-selectin antagonist increased mTOR expression in MSC from AML-PDX. Combination treatment with Compound A and venetoclax/HMA induced higher Ki67 positivity, as well as hyperactivation of pRb and p-S6 in MSC in vivo.

Collectively, the results of Examples 1-3 provide first evidence that an E-selectin targeting strategy with E-selectin antagonists, including but not limited to Compound A, may overcome microenvironmental resistance to venetoclax/HIMA-based therapy in AML by cancer cell autonomous and non-cell autonomous mechanisms (e.g., by disrupting signaling pathways) in the bone marrow vascular niche. Additionally, these results suggest that inhibition of E-selectin may protect bone marrow niches by blocking NO production through reduction of PI3K-AKT-eNOS phosphorylation in endothelial cells and by promoting MSC pro-survival signaling pathways that can support nonmalignant HSC, potentially resulting in faster recovery and longer remission duration following venetoclax/HMA treatment.

### Example 4

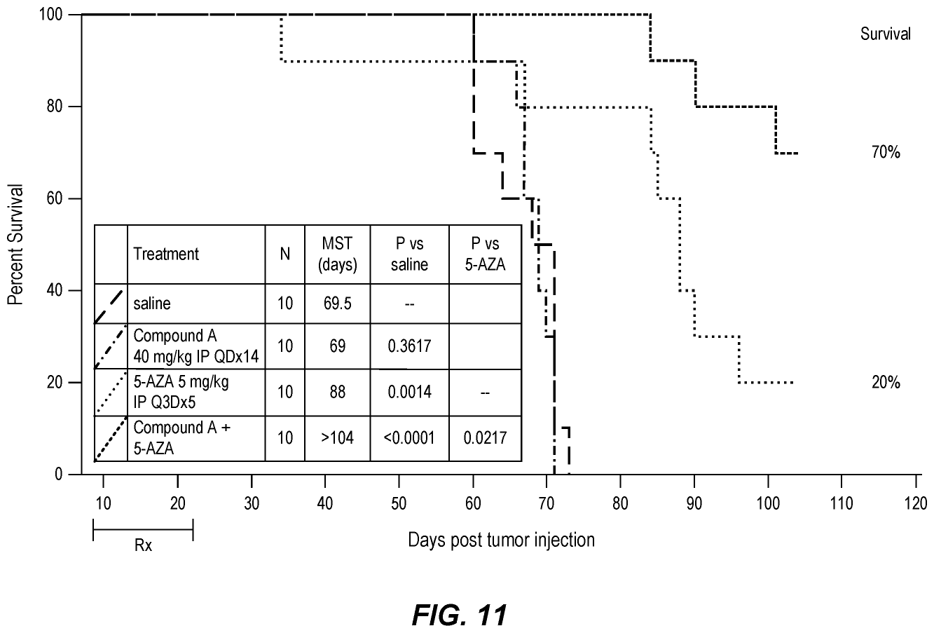

A KG1 AML mouse model was also employed to determine whether the E-selectin antagonist Compound A could enhance the anti-tumor effect of 5-azacitidine. Female NSG mice (10 per cohort, six weeks of age) received i.v. injections of 5×10⁶ KG1 AML tumor cells per mouse. Beginning 7 days post injection, mice were randomized into four cohorts and treated with either saline (i.p. (intraperitoneal), qdx14 (once daily for 14 days)), Compound A (40 mg/kg i.p. qdx14), 5-azacitidine (5 mg/kg i.p. q3dx5), or a combination of Compound A and 5-azacitidine. The efficacies of the treatments on survival were determined by the Kaplan-Meier estimator and log-rank statistics were used to test for significant differences in survival (FIG. 11). The median survival time (MST) of mice treated with 5-azacitidine was 88 days and statistically different from that of mice treated with saline (MST=69.5 days) or Compound A alone (MST=69 days). All mice treated with saline or Compound A alone succumbed to progressive tumor growth. At study conclusion, (day 104 post tumor injection) 20% of mice treated with 5-azacitidine remained alive. Importantly, the therapeutic activity of 5-azacitidine was significantly enhanced when combined with Compound A (MST>104 days, p=0.0140 compared to 5-azacitidine alone). These results suggest that interaction between AML blasts and E-selectin in the KG1 model partially protects leukemia cells from the anti-tumor activity of 5-azacitidine and that Compound A attenuates this protection.

### Example 5

To further explore this hypothesis, the ability of Compound A to disrupt adhesion of KG1 AML cells to E-selectin was assessed using an in vitro assay. Recombinant human E-selectin-Fc chimera was purchased from R&D Systems (724-ES). KG1 AML cell line was purchased from ATCC (CRL-8031) and cultured in RPMI-1640 medium supplemented with 10% fetal bovine serum (FBS). Costar 96-well polystyrene medium binding assay plates were purchased from Corning (9017). 5-azacitidine (5-AZA) was purchased from Sigma-Aldrich (A2386). Calcein AM was purchased from Molecular Probes (C3100MP). FITC-conjugated antibody reactive with cutaneous lymphocyte antigen (HECA-452-FITC) was purchased from BD Pharmingen (555947).

The wells of a 96-well polystyrene plate were coated with 100 μL of 2 μg/mL recombinant human E-selectin-Fc chimera for 2 hours at 37° C., and then washed three times with Hank's Balanced Saline Solution (HBSS). KG1 cells were fluorescently labeled in culture medium with 3 μM Calcein AM for 60 minutes at 37° C., pelleted by centrifugation at 250×g for 10 minutes, then resuspended in HBSS to 2.5×10⁵ cells per mL. Next, 2.5×10⁴ cells were added to each well, and the cells were allowed to adhere for 45 minutes at room temperature. In some cases, cells were treated daily with 100 nM 5-azacitidine for 96 hours prior to labeling with Calcein AM and adhesion to E-selectin. Appropriate wells received 1 μL of 10 mM Compound A (final concentration in well: 100 μM) and after 30 minutes the wells were observed by fluorescence microscopy and pre-wash fluorescence measurements were taken using a FlexStation plate reader (excitation 485 nm, emission 538 nm, cutoff 530 nm). Subsequently, the wells were washed gently three times with HBSS and observations by fluorescence microscopy and fluorescence readings were repeated.

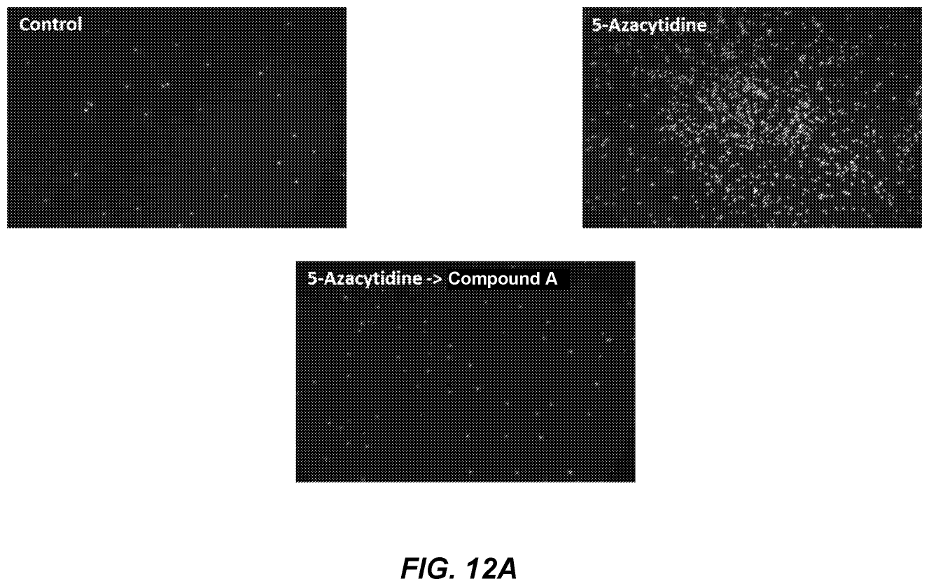

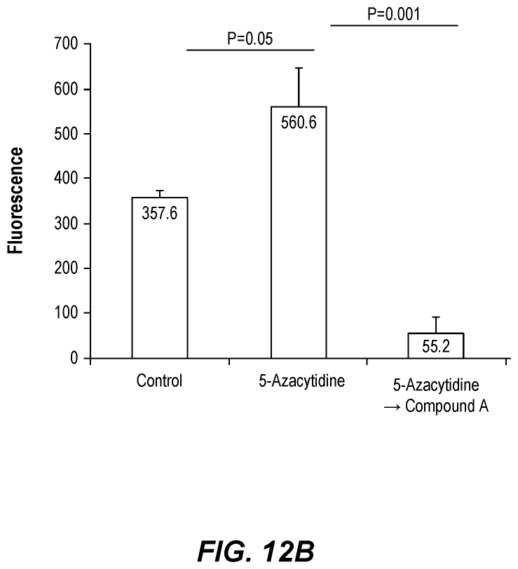

As shown in FIGS. 12A and 12B, incubation of AML cells with 5-AZA enhanced adhesion to E-selectin. The fluorescence units of adhered cells not previously treated with 5-AZA was 357.6, while that of cells treated for 96 hours with 100 nM 5-AZA was 560.6, a 57% increase. Notably, treatment of previously attached cells with Compound A led to significant cellular release (fluorescence units=55.2, p=0.001). These results demonstrate that treatment of the KG1 AML cell line with the hypomethylating reagent 5-AZA enhanced adhesion of the cells to E-selectin and that adhered cells could be released by treatment with the E-selectin antagonist Compound A.

The increased adhesion of KG1 cells to E-selectin following treatment with 5-AZA was further examined by flow cytometry. Cells were cultured for 96 hours in the presence or absence of 100 nM 5-AZA. The binding of E-selectin-PE (E-selectin-Fc chimera conjugated with R-phycoerythrin) to the cells was determined by flow cytometry. In addition, the reactivity of the cells with HECA-452 monoclonal antibody, which specifically reacts with sialyl Lewis A/X carbohydrate structures and is a surrogate marker of E-selectin ligand, was determined by flow cytometry.

Specifically, KG1 cells were centrifuged at 250×g for 10 minutes, washed with HBSS containing 0.1% bovine serum albumin (HBSS/BSA), and resuspended in HBSS/BSA to approximately 3×10⁶ cells per mL. The cells were treated with Fc receptor blocker (Miltenyi Biotech) and 100 μL aliquots (3×10⁵ cells) were added to 12×75 mm Falcon polypropylene tubes. Cells were treated with either 5 μL E-selectin-Fc-PE reagent or 20 μL HECA-452-FITC antibody, placed at 4° C. for 45 minutes, washed with 2 mL then again with 1 mL HBSS/BSA. Final cell pellets were resuspended in 500 μl HBSS/BSA and analyzed on an Attune NxT flow cytometer. E-selectin was conjugated with R-phycoerythrin using the PE/R-phycoerythrin conjugation kit—Lightning-Link (Abcam ab102918).

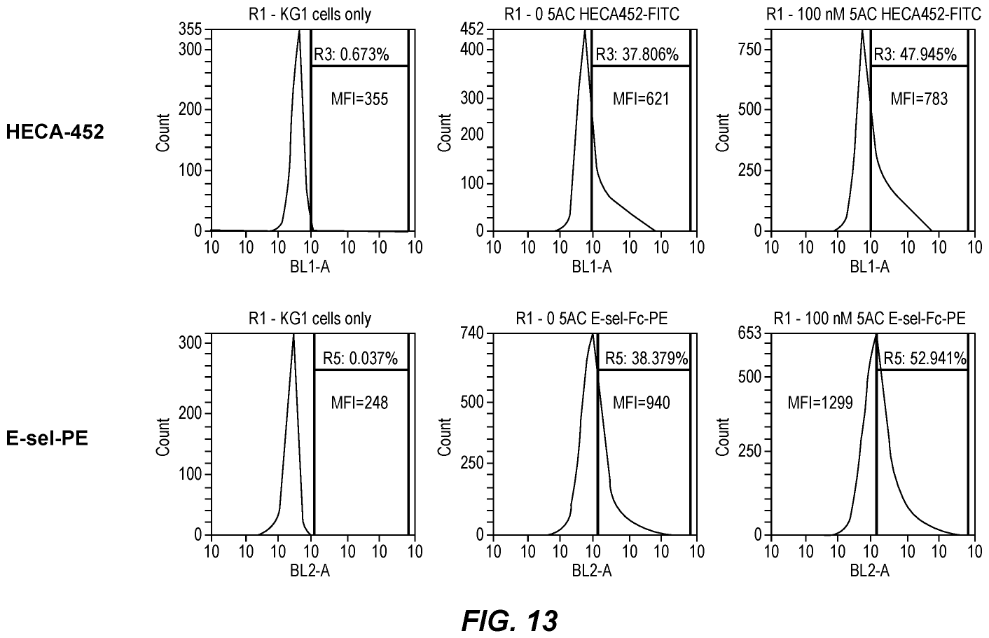

Treatment of cells with 5-AZA increased cell surface expression of E-selectin ligands as demonstrated by increased reactivity with E-selectin-PE and HECA-452-FITC (FIG. 13). Treatment with 5-AZA yielded a 38% increase in both the percentage of cells reactive with E-selectin-PE (38.4% to 52.9%) and in the median fluorescence intensity (MFI, 940 to 1299). Similarly, treatment with 5-AZA resulted in a 27% increase in the percentage of cells reactive with HECA-452 (37.8% to 47.9%) and a 26% increase in MFI (621 to 783).

The observed increase in E-selectin ligands on cell surfaces following treatment with 5-AZA suggests that the hypomethylating activity of 5-AZA may enhance expression of genes encoding enzymes involved in the biosynthesis of sialyl Lewis A/X carbohydrates. Prior to assessing the effect of 5-AZA on specific gene expression, its effect on global DNA methylation was assessed by specifically measuring levels of 5-methylcytosine (5-mC) in a colorimetric ELISA-like reaction. DNA was isolated from cell pellets using a DNA Isolation Kit for Cells and Tissues (Roche Catalog No. 11 814 770 001). DNA was quantified using a DNA Quantification Assay Kit (BioVision Catalog No. K539-200). Global DNA methylation was measured using the MethylFlash™ Global DNA Methylation (5-mC) ELISA Easy Kit (EpiGentek Catalog No. P-1030).

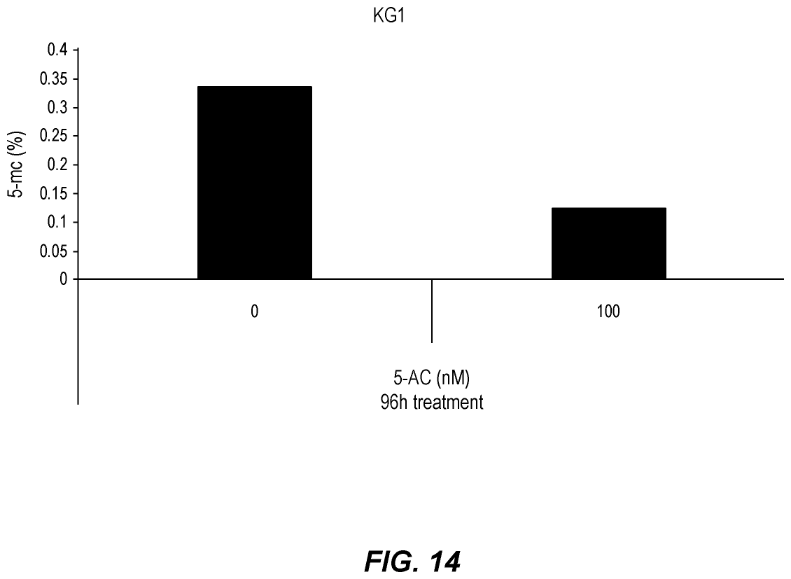

KG1 cells were either treated with vehicle or cultured for 96 hours in the presence of 100 nM 5-AZA. DNA was isolated and purified from cell pellets and evaluated for 5-mC levels. As shown in FIG. 14, the level of 5-mC in untreated KG1 cells was 0.33% while that in cells treated with 5-AZA was 0.12%. This result demonstrates that treatment with 100 nM 5-AZA yielded a substantial hypomethylating effect.

To address the hypothesis that hypomethylation led to enhanced expression of glycosyltransferases, KG1 cells were cultured in the presence or absence of 100 nM 5-AZA for 96 hours followed by real time qPCR analysis of mRNAs encoding relevant glycosyltransferases. Fresh 5-AZA was added to the culture daily. Approximately 1×10⁶ cells were pelleted by centrifugation at 250×g for 10 minutes then snap frozen on dry ice. Total RNA was extracted and purified using a QIAGEN RNeasy® Kit with an on-column DNase treatment step (QIAGEN Cat. No. 74104). The fold-change (2\hat{ }(−Delta Ct)) is the normalized gene expression (2\hat{ }(−Delta Ct)) in the 5-AZA treated sample divided the normalized gene expression (2\hat{ }(−Delta Ct)) in the control sample.

Several genes involved in the biosynthesis of Lewis antigens showed enhanced expression following treatment with 100 nM 5-AZA for 96 hours (Table 1). In Table 1, fold-regulation represents fold-change results in a biologically meaningful way. Fold-change values greater than one indicate a positive- or an up-regulation, and the fold-regulation is equal to the fold-change. Fold-change values less than one indicate a negative or down-regulation, and the fold-regulation is the negative inverse of the fold-change. Additionally, p-values in Table 1 were calculated based on a Student's t-test of the replicate 2\hat{ }(−Delta Ct) values for each gene in the control group and treatment groups.

TABLE 1

| | AVG ΔCₜ | | | | | | Fold Up- |
|---|---|---|---|---|---|---|---|
| | (Ct(GOI) - Ave Ct | | | | | | or Down- |
| | (HKG)) | | 2\hat{ }-ΔCₜ | | Fold Change | | Regulation |
| | Test | Control | Test | Control | Test Sample/ | T-TEST | Test Sample/ |
| Symbol | Sample | Sample | Sample | Sample | Control Sample | p value | Control Sample |
| ST3GAL3 | 10.95 | 11.05 | 5.0E−04 | 4.7E−04 | 1.07 | 0.855872 | 1.07 |
| ST3GAL6 | 5.66 | 7.01 | 2.0E−02 | 7.8E−03 | 2.55 | 0.000018 | 2.55 |
| FUT9 | 14.46 | 15.39 | 4.4E−05 | 2.3E−05 | 1.92 | 0.191653 | 1.92 |
| FUT4 | 14.78 | 14.75 | 3.6E−05 | 3.6E−05 | 0.98 | 0.671717 | −1.02 |
| FUT7 | 11.71 | 15.06 | 3.0E−04 | 2.9E−05 | 10.15 | 0.000040 | 10.15 |
| B3GNT5 | 8.74 | 9.98 | 2.3E−03 | 9.9E−04 | 2.36 | 0.013169 | 2.36 |
| FUT3 | 14.89 | 15.39 | 3.3E−05 | 2.3E−05 | 1.42 | 0.000155 | 1.42 |
| FUT5 | 14.89 | 14.93 | 3.3E−05 | 3.2E−05 | 1.03 | 0.736521 | 1.03 |
| FUT6 | 13.39 | 15.10 | 9.3E−05 | 2.9E−05 | 3.27 | 0.295733 | 3.27 |
| ST3GAL4 | 7.17 | 8.65 | 6.9E−03 | 2.5E−03 | 2.78 | 0.000027 | 2.78 |
| CDKN1C | 10.69 | 10.99 | 6.1E−04 | 4.9E−04 | 1.24 | 0.212095 | 1.24 |
| ACTB | 0.87 | 0.07 | 5.5E−01 | 9.5E−01 | 0.58 | 0.000001 | −1.73 |
| GAPDH | −0.87 | −0.07 | 1.8E+00 | 1.1E+00 | 1.73 | 0.000017 | 1.73 |

FUT7, the gene which encodes a(1,3)-fucosyltransferase VII, an enzyme which catalyzes the last step of sLeX synthesis, was upregulated 10.15-fold (p=0.000040) compared to control samples not treated with 5-AZA. ST3GAL4, which encodes α(2,3)-sialyltransferase IV, the primary sialyltransferase regulating the synthesis of E-selectin ligands on human myeloid cells, was upregulated 2.78-fold (p=0.000027). B3GNT5, which encodes a member of the β(1,3)—N-acetylglucosaminyltransferase family, was upregulated 2.36-fold (p=0.013). Thus, treatment of KG1 cells with 5-AZA upregulated expression of genes encoding enzymes involved in the biosynthesis of the E-selectin ligand sialyl Lewis X.

To test whether the increased expression of FUT7 mRNA in KG1 cells treated with 5-AZA could be due to hypomethylation of the FUT7 promoter, targeted Next-Gen bisulfite sequencing of the FUT7 promoter region was performed. The methylation status of 101 CpG sites surrounding the transcription start site was determined. Specifically, KG1 cells were cultured in the presence of 100 nM 5-AZA, with fresh hypomethylating reagent added to the culture daily. Cells were collected after 96 hours of treatment and cell pellets were prepared. Extracted DNA samples (500 ng) were bisulfite modified using the EZ-96 DNA Methylation-Direct Kit™ (ZymoResearch; Irvine, Calif.; Catalog No. D5023) per the manufacturer's protocol with minor modification. The bisulfite modified DNA samples were eluted using M-elution buffer in 46 μL. Following DNA extraction and bisulfite modification, 26 regions surrounding the transcription start site were evaluated by PCR/NGS to assess the methylation status of 101 CpG sites. All bisulfite modified DNA samples were amplified using separate multiplex or simplex PCRs. PCRs included 0.5 units of HotStarTaq (Qiagen; Hilden, Germany; Catalog No. 203205), 0.2 μM primers, and 3 μL of bisulfite-treated DNA in a 20 μL reaction.

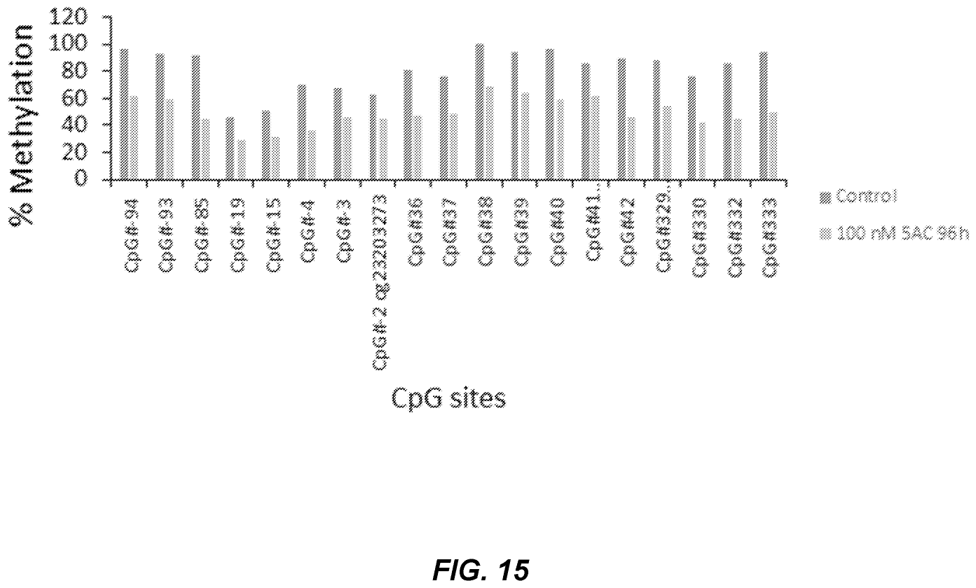

The results (FIG. 15) showed a dose and time dependent demethylation of multiple CpG sites in the region 3928 bp upstream of the transcription start site (TSS) to 6054 bp downstream of the TSS. FIG. 15 highlights the percent methylation of the 19 CpG sites that showed 50% or higher methylation in the absence of 5-AZA treatment. Treatment with 5-AZA resulted in demethylation of these sites, suggesting that hypomethylation of the promoter region resulted in higher expression of FUT7 and subsequently higher levels of the E-selectin ligand sialyl Lewis X on the surface of the KG1 cells.

Together, the data of Examples 4 and 5 indicate that HMA treatment of KG1 AML cells upregulated expression of glycogenes involved in the synthesis of sialyl Lewis X (sLex), the carbohydrate ligand for E-selectin. Not only were higher levels of gene expression observed, but higher levels of the E-selectin ligand were displayed on the cell surface following HMA treatment as evidenced by enhanced reactivity with E-selectin. Thus, when HMA therapy is used in the clinic to treat patients unsuitable for standard of care intensive induction chemotherapy, augmented expression of E-selectin ligands on the leukemic blasts may occur, which could lead to chemoresistance and disease relapse. This scenario underscores the potential utility of E-selectin antagonists such as Compound A for inhibiting blast adhesion to E-selectin on the bone marrow vasculature, hence diminishing chemoresistance and relapse.

### Example 6

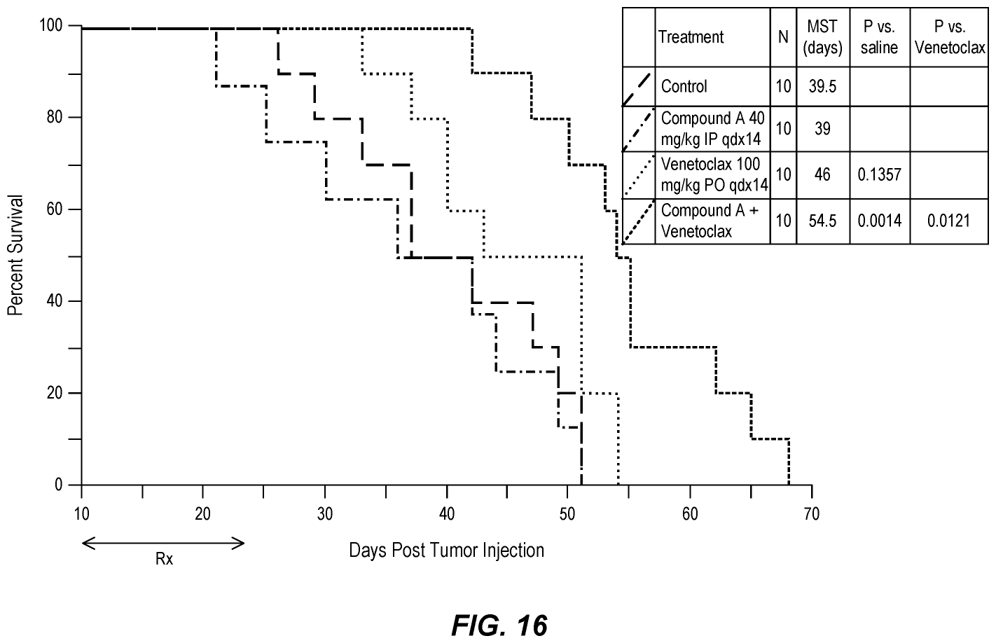

To evaluate the efficacy of targeting E-selectin with Compound A in combination with venetoclax, an in vivo MV4.11 AML model was employed (FIG. 16).

luc-MV4.11 cells (5×10⁶ cells/mouse) were transplanted into NSG mice. Mice were divided into four groups: vehicle treatment only; 40 mg/kg of Compound A (intraperitoneal, 14 day once daily); 100 mg/kg venetoclax (oral, 14 days once daily), and a combination of 40 mg/kg Compound A and 100 mg/kg venetoclax. Drug treatment was initiated on day 10 post-transplantation.

The median survival time (MST) of mice treated with venetoclax alone or venetoclax in combination with Compound A was 46 days or 54.5 days, respectively, both of which were statistically different from that of mice treated with saline (MST=39.5 days) or Compound A alone (MST=39 days).

### REFERENCES

The following references are hereby incorporated by reference in their respective entireties.
C. D. DiNardo et al., “Venetoclax combined with decitabine or azacitidine in treatment-naive, elderly patients with acute myeloid leukemia,” Blood, 133(1): 7-17 (Jan. 3, 2019).
M. Y. Konopleva & C. T. Jordan, “Leukemia Stem Cells and Microenvironment: Biology and Therapeutic Targeting,” J. Clin. Oncol., 29(5): 591-99 (Feb. 10, 2011).
M. Y. Konopleva et al., “Efficacy and Biological Correlates of Response in a Phase II Study of Venetoclax Monotherapy in Patients with Acute Myelogenous Leukemia,” Cancer Discovery, 6(10): 1106-17 (October 2016).
M. P. Bevilacqua et al., “Identification of an inducible endothelial-leukocyte adhesion molecule,” PNAS, 84(24):9238-9242 (1987).
I. Winkler et al., “Vascular niche E-selectin regulates hematopoietic stem cell dormancy, self renewal and chemoresistance,” Nat. Med., 18(11):1651-1657 (2012).
D. S. Krause et al., “Requirement for CD44 in Homing and Engraftment of BCR-ABL-expressing Leukemic Stem Cells,” Nat. Med., 12(10):1175-80 (October 2006).
S. Aref et al., “Soluble Hepatocyte Growth Factor (sHGF) and Vascular Endothelial Growth Factor (sVEGF) in Adult Acute Myeloid Leukemia: Relationship to Disease Characteristics,” Hematology, 7(5):273-279 (2002).
Burnett et al., “Attempts to optimize induction and consolidation treatment in acute myeloid leukemia: results of the MRC AML12 trial,” J. Clin. Oncol. 2010; 28:586-595.
Fernandez et al., “Anthracycline Dose Intensification in Acute Myeloid Leukemia,” N. Engl. J. Med. 2009; 361:1249-1259.
Mandelli et al., “Daunorubicin Versus Mitoxantrone Versus Idarubicin As Induction and Consolidation Chemotherapy for Adults With Acute Myeloid Leukemia: The EORTC and GIMEMA Groups Study AML-10,” J. Clin. Oncol. 2009; 27:5397-5403.
Ravandi et al., “Eradication of Leukemia Stem Cells as a New Goal of Therapy in Leukemia,” Clin. Can. Res. 2006; 12(2):340-344.
Kupsa T. et al., “Serum levels of soluble adhesion molecules in newly diagnosed acute myeloid leukemia and in complete remission suggest endothelial cell activation by myeloblasts,” Biomed Pap Med Fac Univ Palacky Olomouc Czech Repub. 2016; 160:94-99.
Nakao M, Yokota S, Iwai T, et al. “Internal tandem duplication of the flt3 gene found in acute myeloid leukemia,” Leukemia. 1996 December; 10(12):1911-1918.
Kottaridis P. D. et al., “Prognostic Implications of the Presence of FLT3 Mutations in Patients with Acute Myeloid Leukemia,” Leukemia & Lymphoma, 2003; 44:6, 905-913.
Thiede C., et al., Analysis of FLT3-activating mutations in 979 patients with acute myelogenous leukemia: association with FAB subtypes and identification of subgroups with poor prognosis: Presented in part at the 42nd Annual Meeting of the American Society of Hematology, Dec. 1-5, 2000, San Francisco, Calif. (abstract 2334). Blood 2002; 99(12): 4326-4335.
Falini et al., New Classification of Acute Myeloid Leukemia and Precursor-related Neoplasms: Changes and Unsolved Issues, Discov. Med. 2010; 10(53):281-92.
Lee et al., Minimally Differentiated Acute Nonlymphocytic Leukemia: A Distinct Entity, Blood 1987; 70(5):1400-1406.

Those of ordinary skill in the art will recognize or be able to ascertain using no more than routine experimentation, many equivalents to the specific embodiments of the disclosure described herein. Such equivalents are intended to be encompassed by the following claims.

1. A method of treating a cancer in a subject in need thereof comprising administering to the subject at least one E-selectin antagonist, wherein the subject is further administered at least one antineoplastic agent.

2. (canceled)

3. The method according to claim 1, wherein the subject is further administered at least one hypomethylating agent.

4. The method according to claim 3, wherein the at least one hypomethylating agent is chosen from 5-azacitidine, 5-aza-2′-deoxycytidine (decitabine), guadecitabine, 5-fluoro-2′-deoxycytidine, zebularine, CP-4200, RG108, and nanaomycin A.

5. The method according to claim 3, wherein the at least one hypomethylating agent is 5-azacitidine.

6. The method according to claim 3, wherein the at least one hypomethylating agent is decitabine.

7. The method according to claim 3, wherein the at least one antineoplastic agent is chosen from targeted therapy drugs.

8. The method according to claim 3, wherein the at least one antineoplastic agent is venetoclax.

9. The method according to claim 8, wherein the method comprises administering to the subject a fixed dose of 10 mg to 1000 mg per day of venetoclax.

10. The method according to claim 3, wherein the at least one antineoplastic agent is chosen from chemotherapeutic agents.

11. The method according to claim 1, wherein the at least one E-selectin antagonist is chosen from carbohydrate mimetics of an E-selectin ligand.

12. The method according to claim 1, wherein the at least one E-selectin antagonist is chosen from 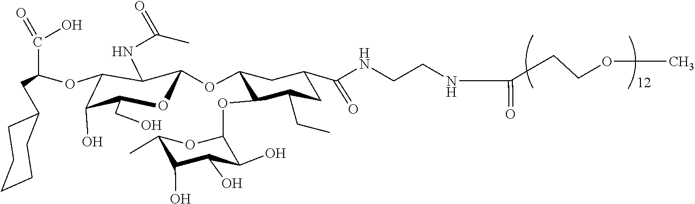
and pharmaceutically acceptable salts thereof.

13. The method according to claim 12, wherein the method comprises administering to the subject a fixed dose of 20 mg to 4000 mg per day of the at least one E-selectin antagonist.

14. The method according to claim 12, wherein the cancer is chosen from liquid cancers.

15. The method according to claim 12, wherein the cancer is chosen from solid cancers.

16. The method according to claim 12, wherein the cancer is chosen from FLT3 mutated cancers.

17. The method according to claim 12, wherein the cancer is chosen from FLT3-ITD mutated cancers.

18. The method according to claim 12, wherein the cancer is chosen from colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, breast cancer, pancreatic cancer, leukemia, lymphoma, myeloma, melanoma, kidney chromophobe carcinoma, adrenocortical carcinoma, bladder urothelial carcinoma, thymoma, testicular germ cell tumors, and head and neck squamous cell carcinoma.

19. The method according to claim 12, wherein the cancer is chosen from melanoma, leukemia, kidney chromophobe carcinoma, adrenocortical carcinoma, bladder urothelial carcinoma, lymphoma, thymoma, testicular germ cell tumors, and head and neck squamous cell carcinoma.

20. The method according to claim 19, wherein the leukemia is chosen from acute myeloid leukemia, acute lymphocytic leukemia, chronic lymphocytic leukemia, and chronic myelogenous leukemia.

21. The method according to claim 19, wherein the lymphoma is chosen from non-Hodgkin's lymphoma and Hodgkin's lymphoma.

22. The method according to claim 19, wherein the myeloma is multiple myeloma.

23. The method according to claim 19, wherein the melanoma is chosen from uveal melanoma and skin melanoma.

24. The method according to claim 1, wherein the subject has acquired resistance to a therapy comprising at least one antineoplastic agent.

25. The method according to claim 3, wherein the subject has acquired resistance to a therapy comprising at least one hypomethylating agent.

26. The method according to claim 3, wherein the subject has acquired resistance to a combination therapy comprising at least one antineoplastic agent and at least one hypomethylating agent.

27. The method according to claim 1, wherein the subject possesses one or more mutational alterations of FLT3.

28. The method according to claim 1, wherein the subject expresses the gene ST3GAL4 at an expression level greater than that of at least 55% of cancer patients.

29. The method according to claim 1, wherein the subject expresses the gene B3GNT5 at an expression level greater than that of at least 55% of cancer patients.

30. The method according to claim 1, wherein the subject expresses the gene FUT7 at an expression level greater than that of at least 55% of cancer patients.

31. The method according to claim 1, wherein the method further comprises selecting the subject to treat through a method comprising: (a) determining or having determined the gene expression level of one or more genes in the subject or a sample from the subject; and (b) selecting the subject for treatment when at least 10% of the blast cells from the subject or sample from the subject expresses the one or more genes.

32. The method according to claim 31, wherein the one or more genes are chosen from ST3GAL4, B3GNT5, and FUT7.

33. The method according to claim 1, wherein the method further comprises selecting the subject to treat through a method comprising: (a) obtaining or having obtained a biological sample comprising blast cells from the subject; (b) performing or having performed an assay on the biological sample to determine the gene expression level of one or more E-selectin ligand-forming genes in the sample; and (c) selecting the subject for treatment when at least 10% of the blast cells in the sample express the one or more E-selectin ligand-forming genes.

34. The method according to claim 33, wherein the one or more E-selectin ligand-forming genes are glycosylation genes.

35. The method according to claim 34, wherein the one or more E-selectin-ligand forming genes are chosen from ST3GAL4 and FUT7.

36. The method according to claim 1, wherein the method further comprises selecting the subject to treat through a method comprising: (a) determining the gene expression level of one or more genes in the subject or a sample from the subject; (b) comparing the gene expression level from (a) to a control sample from a cancer-free subject, a newly diagnosed cancer subject, or a subject diagnosed with the same cancer as the subject, and (c) selecting the subject for treatment when the gene expression level exceeds that in the control sample.

37. The method according to claim 36, wherein the one or more genes are chosen from ST3GAL4, B3GNT5, and FUT7.

38. The method according to claim 1, wherein the administration extends the number of days the subject is in remission, reduces the number of days until remission, inhibits the metastasis of cancer cells, or improves survival.

39. The method according to claim 1, wherein the subject is human.

40. The method according to claim 1, wherein the at least one antineoplastic agent is venetoclax.

41. The method according to claim 4, wherein the at least one antineoplastic agent is venetoclax.

42. The method according to claim 5, wherein the at least one antineoplastic agent is venetoclax.

43. The method according to claim 6, wherein the at least one antineoplastic agent is venetoclax.

44. The method according to claim 4, wherein the at least one antineoplastic agent is venetoclax and wherein the at least one E-selectin antagonist is chosen from 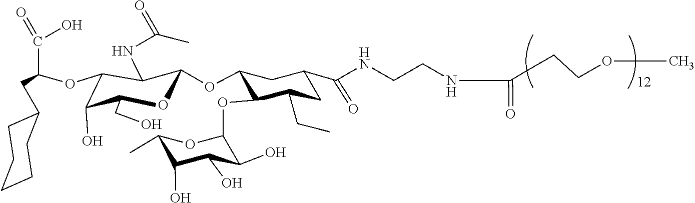
and pharmaceutically acceptable salts thereof.

45. The method according to claim 44, wherein the at least one E-selectin antagonist is the sodium salt of [chemical structure]
